(19) United States
(12) Patent Application Publication
(10) Pub. No.: US 2009/0004207 A1
(43) Pub. Date: Jan. 1, 2009

(54) Methods and Compositions for Inhibiting Pathological Angiogenesis in the Eye

(76) Inventors: Timothy Tun Hla, Avon, CT (US); Athanasia Skoura, West Hartford, CT (US); Bongnam Jung, Farmington, CT (US)

Correspondence Address: CANTOR COLBURN, LLP, 20 Church Street, 22nd Floor, Hartford, CT, 06103, US

(21) Appl. No.: 12/135,406

(22) Filed: Jun. 9, 2008

Related U.S. Application Data
(60) Provisional application No. 60/933,947, filed on Jun. 8, 2007.

(51) Int. Cl. A61K 39/395; A61K 31/7052; A61K 31/437; C12Q 1/02; A61K 48/00; A61P 27/02

(52) U.S. Cl. 424/172.1; 514/44; 514/303; 435/29

(57) ABSTRACT

Disclosed herein are compositions and methods for inhibiting abnormal angiogenesis in the eye, particularly in the retina. The methods include administering to subject in need of treatment for pathological angiogenesis of the eye a pharmaceutically effective amount of an inhibitor of the receptor activity of the S1P2 receptor. Also included are compositions including an S1P2 receptor antagonist and an opthalmically acceptable excipient.

### CROSS-REFERENCE TO RELATED APPLICATIONS

This application claims the benefit of U.S. Patent Application Ser. No. 60/933,947, filed Jun. 8, 2007, which is incorporated by reference herein in its entirety.

### STATEMENT REGARDING FEDERALLY SPONSORED RESEARCH & DEVELOPMENT

This invention was made with support from the United States Government under Grant # HL67330 and Grant # HL70694 from the National Institutes of Health. The U.S. Government has certain rights in the invention.

### BACKGROUND

Sphingosine 1-phosphate (S1P) is a lipid mediator that regulates various biological processes, such as cell proliferation, migration, survival and differentiation. S1P, generated by the phosphorylation of sphingosine by sphingosine kinase 1 (Sphk1) and 2 (Sphk2), is degraded by S1P-specific phosphatases and a lyase. It is a high affinity ligand for five G protein coupled S1P receptors on the cell-surface, S1P₁R, S1P₂R, S1P₃R, S1P₄R and S1P₅R, that regulate distinct intracellular signaling pathways. S1P₁, S1P₂ and S1P₃ receptors are widely expressed, whereas S1P₄ and S1P₅ expression is prominent in cells of the immune and nervous systems, respectively. The S1P₁ receptor couples exclusively to Gᵢ signaling pathway, whereas S1P₂ and S1P₃ receptors couple to Gᵢ as well as to the G_{q} and G_{12/13} pathways. However, S1P₂ activates G_{12/13} potently, whereas S1P₃ activates G_{q} preferentially.

S1P receptors regulate important physiological functions of the vascular system, such as vascular morphogenesis and maturation, cardiac function, vascular permeability and tumor angiogenesis. Indeed, S1P₁ null embryos die due to massive hemorrhage at E12.5-14.5 days of gestation since the S1P₁ receptor is essential for proper stabilization of the embryonic vascular system by promoting the formation of strong N-cadherin-based junctions between endothelial and vascular smooth muscle cells. However, mice that lack either the S1P₂ or the S1P₃ receptor are viable and fertile. Interestingly, S1p1/S1p2 double null embryos showed a more severe phenotype than S1p1 single null embryos, suggesting that S1P₂ receptor is also significant during embryonic vascular development. In addition, S1p2 null mice are profoundly deaf due to vascular abnormalities in stria vascularis of inner ear and degeneration of sensory hair cells of the organ of Corti. Moreover, a mutation in the zebrafish gene miles-apart (Mil), an S1p2 ortholog, results in cardiac developmental defects (cardia bifida) due to defective migration of cardiomyocyte precursors, underscoring the significance of this receptor for the fish cardiac development. However, the role of S1P₂ receptor in vascular development and pathology is virtually unknown.

### SUMMARY

In one embodiment, a method of inhibiting pathological angiogenesis in the eye of a subject in need thereof, comprises administering to the subject a pharmaceutically effective amount of an inhibitor of the receptor activity of the S1P2 receptor.

In another embodiment, a composition suitable for ophthalmic administration comprises an inhibitor of the receptor activity of the S1P2 receptor and an opthalmically acceptable excipient,

wherein the inhibitor is a small molecule of formula J:

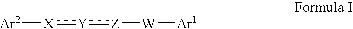

wherein

Ar¹ is an optionally substituted heterocycle or aromatic heterocycle;

Ar² is an optionally substituted heterocycle or aromatic heterocycle;

W is —NR^{a}—, O, or —CH₂— wherein R^{a} is hydrogen or C₁-C₃ alkyl;

Z is —C(═O)—, —C(═S)—, O, —CH₂—, ═N—, or ═CH—;

Y is —NR^{a}—, —C(═O)—, —N═, —CH═, ═N—, or ═CH—; and

X is —NR^{a}—, —N═, —CH═, or —CH₂—.

In yet another embodiment, a method of screening candidate molecules as potential inhibitors of an S1P2 receptor comprises contacting S1P2R-expressing retinal endothelial cells in culture with a candidate molecule, measuring the increase and/or induction of vascular or paracellular permeability in the retinal endothelial cells, determining if the candidate molecule is an inhibitor of an S1P2 receptor, and producing the molecule.

### BRIEF DESCRIPTION OF THE FIGURES

FIG. 1 shows a schematic representation of the hypoxia-induced mouse model of Retinopathy of Prematurity (ROP).

FIG. 2 shows S1P₁, S1P₂, S1P₃ mRNA expression normalized to cyclophilin A mRNA and expressed as fold induction over the relative gene expression value measured at P12 (Hypoxia-Day 0), determined by quantitative RT-PCR analysis (n=3).

FIG. 3 shows Ang-2 and VEGF mRNA in the course of hypoxia (n=3).

FIG. 4 shows that S1p2^{-/-} retinas display increased intraretinal vascularization during the course of hypoxia.

FIG. 5 shows that S1p2^{-/-} retinas display decreased intravitreal neovascularization during the course of hypoxia.

FIG. 6 shows quantification of fluorescent pixels representing BrdU positive cells per retina at P14. S1p2^{+/-} retinas show 0.05±0.006 (n=5) while S1p2^{-/-} retinas show 0.05±0.011 (n=5; *P=0.47) fluorescent pixels/total retinal area, indicating that the S1P2 receptor modulates retinal vascular patterning.

FIG. 7 shows that at the early stage of retina pathogenesis (P15), S1p2^{+/-} retinas develop 23.122±5.73 tip cells/field (n=9) while S1p2^{-/-} retinas develop 34.34±6.3 tip cells/field (n=7; *P<0.0025), indicating that the S1P2 receptor modulates retinal vascular patterning.

FIG. 8 shows that S1P2 regulates inflammatory response in ischemic retinas. At P15, the mean number of F4/80 positive cells is 30.57±8.23 (n=2) for S1p2^{+/+} retinas, 33.91±9.5 (n=4) for S1p2^{+/-} retinas and 16.85±4.33 (n=4; *P<0.02) for S1p2^{-/-} retinas.

FIG. 9 shows that at P17, the mean number of F4/80 positive cells is 83.76±20.93 (n=3) for S1p2^{+/+} retinas and 3 1.81±9.88 (n=3; *P<0.02) for S1p2-retinas.

FIG. 10 shows relative VEGF, Ang-2, Flt1, TNF-a, iNOS, COX-2 mRNA expression in HT and KO ischemic retinas at P13 (24 hours of hypoxia) as determined by quantitative RT-PCR (n=3, *P<0.04). Gene expression is normalized to cyclophilin A expression and expressed as fold induction over the control HT animals.

FIG. 11 shows fold induction of relative COX-2 mRNA expression during the course of hypoxia (n=3, *P<0.02).

FIG. 12 shows immunoblotting for COX-2, S1P₂-V5 and actin expression in HUVECs transduced with AdS1P₂-V5 and AdGFP.

FIG. 13 shows induction of promoter activity of the human COX-2 gene. phPES2(−1432/+59) luciferase reporter (0.3 μg) was cotransfected in EOMA cells with pcDNA 3.1 (control, 0.3 μg), pcDNA3.1-S1p2 receptor plasmid (0.3 μg) or pcDNA3.1-S1p1 receptor plasmid (0.3 μg). Cells transfected with phPES2(−1432/+59) luciferase reporter were treated with PMA (100 nM, positive control). Results from one representative experiment is shown (*P<0.01, **P=0.43, n=4).

FIG. 14 shows eNOS and actin protein expression in HT and KO retinas at P14 (2 days of hypoxia), HUVECs extract as positive control. Increased e.NOS expression by 1.6-fold in KO retinas (*P≦0.05) was observed.

FIG. 15 shows Western blot analysis of extracts from HUVECs transduced with AdGFP or Ad51P₂-V5 (20 MOI). Immunoblotting for eNOS, S1P2-V5 and actin expression. Decreased levels of eNOS in HUVECs transduced with Ad51P₂-V5, by 2.3-fold (**P<0.02, results from one representative experiment, n=5).

FIG. 16 shows HUVEC grown on transwell filters pretreated with vehicle (C) or JTE013 (0.2 μmol/L) (JTE) for 30 minutes. Paracellular permeability after 1 hour was quantified. Where indicated (+S1P), 100 nmol/L S1P was added. Results are mean±SE of triplicates. n=3. *P<0.05 S1P-treated vs nontreated. #P<0.05 JTE pretreated vs. vehicle pretreated.

FIG. 17 shows lungs perfused with either vehicle (Control) or 0.5 μmol/L JTE013 (JTE013) during 15 minutes and K_{f} measurements were taken (BL). Then, 50 μmol/L H₂O₂ was added to the reservoir, allowed to circulate for 15 minutes, and Kf was measured (H₂O₂). Results are mean±SE, n=4. *P<0.01 before vs. after H₂O₂-treatment, #P<0.01 JTE013-treated vs. vehicle-treated.

FIG. 18 shows bovine retinal endothelial cells stimulated with S1P (100 nM), JTE103 (1 μM), VPC44116 (1 μM) or together and analyzed in the Boyden chamber assay to analyze cell migration.

These and other embodiments, advantages and features of the present invention become clear when detailed description and examples are provided in subsequent sections.

### DETAILED DESCRIPTION

Disclosed herein are compositions and methods for inhibiting abnormal angiogenesis in the eye, particularly in the retina. Also provided herein are methods for treating or preventing certain types of blindness. Further provided are compositions comprising a S1P2 receptor antagonist and an opthalmically acceptable excipient.

Sphingosine-1-phosphate (S1P) is a multifunctional lipid mediator that signals via the S1P family of G protein-coupled receptors (S1PR). S1P is known to regulate vascular maturation, permeability and angiogenesis. By example, S1P is known to be a stimulator of angiogenesis, i.e., new blood vessel growth. As used herein, the terms S1P2R, S1P₂R, S1P2 receptor and S1P2 receptor are used interchangeably to mean the sphingosine-1-phosphate receptor 2.

Angiogenesis is directly involved in a number of pathological conditions such as tumor growth, inflammation and diabetic retinopathy. Current approaches to the treatment of abnormal angiogenesis in the eye include laser therapy, which destroys some retinal tissue in order to preserve some vision, and the administration of ant-VEGF antibody and/or ant-VEGF RNA aptomer. There remains a clear need for improved methods and agents for prevention and treatment of conditions involving abnormal angiogenesis and harmful angiogenesis such as pathological angiogenesis in the tissues of the eye.

In order to investigate the role of the S1P2 receptor in mammalian vascular development, the inventors herein studied the retinal vascular development of mice lacking the S1P2 receptor under physiological (normal retina development) and pathophysiological conditions (ischemia-driven retinopathy). Postnatal vascular development of the mouse retina provides an attractive model system to explore the mechanisms of angiogenesis and vascular stabilization. After birth, endothelial cells emerge from the optic disc and form the primary vasculature of the mouse retina. Growing vessels with radial orientation are formed along the retina neuronal and astrocytic plexus. On the other hand, pathological retina angiogenesis produces abnormally growing and chaotically oriented dysfunctional vessels that grow into the vitreous as “vascular tufts” and eventually lead to vision loss. This phenotype is common in the pediatric retinopathy of pematurity (ROP) condition and in diabetic retinopathy of the adult.

It is shown herein that the angiogenic process proceeds normally in S1p2^{-/-} mice during normal retinal development. However, when mice were exposed to ischemic stress, S1p2^{-/-} retinas appear to have increased “physiological” intraretinal angiogenesis and reduced “pathological” intravitreal neovascularization. It was further demonstrated that the S1P₂ receptor is required for the inflammatory cell infiltration, induction of the pro-inflammatory and pro-angiogenic enzyme cyclooxygenase (COX)-2 and the suppression of the endothelial nitric oxide synthase (eNOS) which produces the vasodilator oxide (NO). This study identified S1P signaling by the S1P₂ receptor as a novel target for the prevention and/or treatment of vision-threatening retinopathies.

In one embodiment, a method of treating abnormal angiogenesis in the eye comprises administering to an individual in need thereof an effective amount of an S1P₂ receptor antagonist. As used herein, the term treating includes administration to an individual suffering from abnormal angiogenesis of the eye and administration preventatively or prophylactically to an individual at risk of abnormal angiogenesis of the eye. Administration to an individual at risk of abnormal angiogenesis of the eye can prevent abnormal angiogenesis of the eye. In one embodiment, the individual is at risk of, or has been diagnosed with, abnormal angiogenesis of the eye.

In one embodiment, pathological angiogenesis in the eye is associated with an ocular neovascular disease. This type of disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens disease, acne rosacea, phylectenulosis, syphilis, Mycobacteria infections, lipid degeneration, chemical burns, bacterial ulcers, fungal ulcers, Herpes simplex infection, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's sarcoidosis, scleritis, Stevens-Johnson's disease, pemphigoid, and radial keratotomy.

Diseases associated with retinal/choroidal neovascularization include, but are not limited to, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacteria infections, lyme disease, systemic lupus erythematosis, retinopathy of prematurity, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other eye-related diseases include, but are not limited to, diseases associated with rubeosis (neovascularization of the angle) and diseases caused by the abnormal proliferation of fibrovascular or fibrous tissue, including all forms of prolific vitreoretinopathy.

In another embodiment, pathological angiogenesis of the eye is associated with a neoplastic eye disease. Neoplastic eye diseases include primary ocular tumors, such as uveal melanomas, melanocytomas, retinocytomas, retinal hamartomas and choristomas, retinal angiomas, retinal gliomas and astocytomas, choroidal hemangiomas, choroidal neurofibromas, choroidal hamartomas and choristomas, ocular lymphomas and ocular phakomatoses; and metastatic ocular tumors related to choroidal and retinal neovascularization. Similar to the non-neoplastic diseases, the above tumors also share the retinal neovascularization as a key component.

In one embodiment, disclosed herein is a method of treating an eye injury, comprising locally administering an effective amount of an agent capable of blocking or inhibiting an S1P₂ receptor in a subject in need thereof, such that the eye injury is ameliorated or improved. In one embodiment, the injury is a retinal injury. In another embodiment, the eye injury is a corneal injury or conjunctival injury. In one embodiment, the method of treatment reduces angiogenesis and inflammation associated with the eye injury. In various embodiments, the eye injury is caused by trauma, e.g., surgical injuries, chemical burn, corneal transplant, infectious or inflammatory diseases.

The terms “blocker”, “inhibitor”, or “antagonist” are used interchangeably to mean a substance that retards or prevents a chemical or physiological reaction or response. Exemplary blockers or inhibitors comprise, but are not limited to, antisense molecules, siRNA molecules, antibodies, small molecule antagonists and their derivatives. An S1P₂ receptor blocker or inhibitor inhibits the activity and/or concentration of an S1P₂ receptor. An S1P₂ receptor blocker or inhibitor is an S1P₂ receptor antagonist such as a small molecule, an antibody, an antisense nucleic acid or an siRNA.

In one embodiment, the S1P₂ receptor antagonist is a small molecule such as a molecule of Formula J:

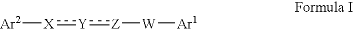

wherein

Ar¹ is optionally substituted heterocycle or aromatic heterocycle;

Ar² is optionally substituted heterocycle or aromatic heterocycle;

W is —NR^{a}—, O, or —CH₂—, wherein R^{a} is hydrogen or C₁-C₃ alkyl;

Z is —C(═O)—, —C(═S)—, O, —CH₂—, ═N—, or ═CH—;

Y is —NR^{a}—, —C(═O)—, —N═, —CH═, ═N—, or ═CH—; and

X is —NR^{a}—, —N═, —CH═, or —CH₂—. When substituted, the substituents on Ar¹ and Ar² include halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

Specifically, exemplary antagonists include those of Formula II wherein

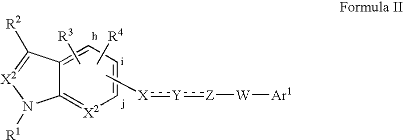

Ar¹ is aromatic heterocycle;

W, Z, Y and X are as previously defined;

R¹ is C₁-C₁₂ alkyl;

R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;

R³ and R⁴ can be positioned at h, i, or j, but not simultaneously at the same position; and

X² is N or —CR^{b}—wherein R^{b} is hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy.

More specifically, exemplary antagonists include those of Formula III wherein

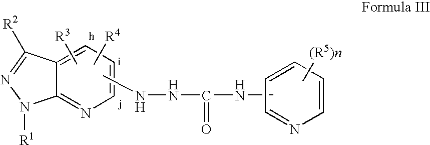

R¹, R², R³, and R⁴ are as previously defined;

each instance of R⁵ is halogen, C₁-C₆ alkyl, C₁-C₄alkoxy, C₁-C₆ perhaloalkyl, C₁-C₄ perhaloalkoxy, amino, mono- or di- C₁-C₄alkylamino, C₃-C₇cycloalkyl, or C₃-C₇cycloalkyloxy; and n is 0, 1, 2, 3, or 4.

In a specific embodiment, antagonists include those of Formula III wherein R¹ is C₁-C₃ alkyl; R² is C₁-C₃ alkyl; R³ is at position h and is C₁-C₆ alkyl; R⁴ is hydrogen; R⁵ is halogen; and n is 2.

Additional exemplary antagonists include 1-[1,3-dimethyl-4-(2-methylethyl)-1H-pyrazolo[3,4-b]pyridin-6-yl]-4-(3,5-dichloro-4-pyridinyl)-semicarbazide (“JTE 013”; CAS No. [547756-93-4]); 1-[1,3-dimethyl-4-(2-methylethyl)-1H-pyrazolo[3,4-b]pyridin-6-yl]-4-(3,5-difluoro-4-pyridinyl)-semicarbazide; 1-[1,3-dimethyl-4-(2-methylethyl)-1H-pyrazolo[3,4-b]pyridin-6-yl]-4-(3,5-dibromo-4-pyridinyl)-semicarbazide; 1-[1,3-dimethyl-4-(2-methylethyl)-1H-pyrazolo[3,4-b]pyridin-6-yl]-4-(3,5-dichloro-4-phenyl)-semicarbazide; 1-[1,3-dimethyl-4-(2-methylethyl)-1H-pyrazolo[3,4-b]pyridin-6-yl]-4-(3,5-difluoro-4-phenyl)-semicarbazide; 1-[1,3-dimethyl-4-(2-methylethyl)-1H-pyrazolo[3,4-b]pyridin-6-yl]-4-(3,5-dibromo-4-phenyl)-semicarbazide; 1-[1,3-dimethyl-4-(2-methylethyl)-1H-pyrazolo[3,4-b]pyridin-6-yl]-4-(3,5-dimethoxy-4-pyridinyl)-semicarbazide; 1-[1,3-dimethyl-4-(2-methylethyl)-1H-pyrazolo[3,4-b]pyridin-6-yl]-4-(3,5-dimethyl-4-pyridinyl)-semicarbazide; 1-[1,3-dimethyl-4-(2-methylethyl)-1H-pyrazolo[3,4-b]pyridin-6-yl]-4-(3-chloro,5-fluoro-4-pyridinyl)-semicarbazide; 1-[1,3-dimethyl-4-(2-methylethyl)-1H-pyrazolo[3,4-b]pyridin-6-yl]-4-(3,5-dimethoxy-4-phenyl)-semicarbazide; 1-[1,3-dimethyl-4-(2-methylethyl)-1H-pyrazolo[3,4-b]pyridin-6-yl]-4-(3,5-dimethyl-4-phenyl)-semicarbazide; 1-[1,3-dimethyl-4-(2-methylethyl)-1H-pyrazolo[3,4-b]pyridin-6-yl]-4-(3-chloro, 5-fluoroo-4-phenyl)-semicarbazide.

Exemplary antagonists include the pyrazolopyridine and related compounds disclosed in WO 01/98301 to Kawasaki et al., incorporated herein by reference in its entirety.

The active agents can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment, an S1P₂ receptor inhibitor is an antibody. The present disclosure includes isolated (i.e., removed from their natural milieu) antibodies that selectively bind an S1P₂ receptor. As used herein, the term “selectively binds to” refers to the ability of antibodies of the present disclosure to preferentially bind to an S1P₂ receptor. Binding can be measured using a variety of methods standard in the art including enzyme immunoassays (e.g., ELISA), immunoblot assays, and the like; see, for example, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999. An antibody selectively binds to or complexes with an S1P₂ receptor, preferably in such a way as to reduce the activity of an S1P₂ receptor.

As used herein, antibody includes antibodies in serum, or antibodies that have been purified to varying degrees, specifically at least about 25%. The antibodies are specifically purified to at least about 50% homogeneity, more specifically at least about 75% homogeneity, and most specifically greater than about 90% homogeneity. Antibodies may be polyclonal antibodies, monoclonal antibodies, humanized or chimeric antibodies, anti-idiotypic antibodies, single chain antibodies, Fab fragments, fragments produced from an Fab expression library, epitope-binding fragments of the above, and the like. An antibody includes a biologically active fragment, that is, a fragment of a full-length antibody the same target as the full-length antibody. Biologically active fragments include Fab, F(ab′)₂ and Fab′ fragments.

Antibodies are prepared by immunizing an animal with full-length polypeptide or fragments thereof. The preparation of polyclonal antibodies is well known in the molecular biology art; see for example, Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology, (1992).

A monoclonal antibody composition is produced, for example, by clones of a single cell called a hybridoma that secretes or otherwise produces one kind of antibody molecule. Hybridoma cells are formed, for example, by fusing an antibody-producing cell and a myeloma cell or other self-perpetuating cell line. Numerous variations have been described for producing hybridoma cells.

In one embodiment, monoclonal antibodies are obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3×63-Ag8.653, Sp2/0-Ag14) and the like. Treatment with the transforming agent results in production of a hybridoma by means of fusing the suspended spleen cells with, for example, mouse myeloma cells. The transformed cells are then cloned, preferably to monoclonality. The cloning is performed in a medium that will not support non-transformed cells, but that will support transformed cells. The tissue culture medium of the cloned hybridoma is then assayed to detect the presence of secreted antibody molecules by antibody screening methods known in the art. The desired clonal cell lines are then selected.

A therapeutically useful antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions.

In addition, chimeric antibodies can be obtained by splicing the genes from a mouse antibody molecule with appropriate antigen specificity together with genes from a human antibody molecule of appropriate biological specificity. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies are used to produce single chain antibodies, as described, for example, in U.S. Pat. No. 4,946,778. Single chain antibodies are formed by linking the heavy and light chain fragments of the Fv region via an amino acid bridge, resulting in a single chain polypeptide.

In one embodiment, antibody fragments that recognize specific epitopes are generated by techniques well known in the art. Such fragments include Fab and F(ab′)₂ fragments produced by proteolytic digestion, and Fab′ fragments generated by reducing disulfide bridges. Fab, F(ab′)₂ and Fab′ fragments of antibodies can be prepared. Fab fragments are typically about 50 kDa, while F(ab′)₂ fragments are typically about 100 kDa in size. Antibodies are isolated (e.g., on protein G columns) and then digested and purified with sepharose coupled to papain and to pepsin in order to purify Fab and F(ab′)₂ fragments according to protocols provided by the manufacturer (Pierce Chemical Co.). The antibody fragments are further purified, isolated and tested using ELISA assays. Antibody fragments are assessed for the presence of light chain and Fc epitopes by ELISA.

In another embodiment, antibodies are produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic subjects are known in the art.

The structure of the antibody may also be altered by changing the biochemical characteristics of the constant regions of the antibody molecule to a form that is appropriate to the particular context of the antibody use. For example, the isotype of the antibody may be changed to an IgA form to make it compatible with oral administration. IgM, IgG, IgD, or IgE isoforms may have alternate values in the specific therapy in which the antibody is used.

Antibodies are purified by methods known in the art. Suitable methods for antibody purification include purification on Protein A or Protein G beads, protein chromatography methods (e.g., DEAE ion exchange chromatography, ammonium sulfate precipitation), antigen affinity chromatography and others.

In one embodiment, a monoclonal antibody that acts as an S1P₂ receptor inhibitor is Sphingomab developed by Lpath, Inc. A monoclonal antibody against the S1P2 receptor is used alone or in combination with other S1P2R inhibitors and regulating agents disclosed herein.

In one embodiment, the S1P2 receptor antagonist comprises an antisense RNA. An antisense RNA (aRNA) is single-stranded RNA that is complementary to a messenger RNA (mRNA) strand transcribed within a cell. Antisense RNA may be introduced into a cell to inhibit translation of a complementary mRNA by base pairing to it and physically obstructing the translation machinery. An antisense molecule specific for an S1P2 receptor should generally be substantially identical to at least a portion, specifically at least about 20 continuous nucleotides, of the nucleic acid encoding the S1P2 receptor, but need not be identical. The antisense nucleic acid molecule can be designed such that the inhibitory effect applies to other proteins within a family of genes exhibiting homology or substantial homology to the nucleic acid. The introduced antisense nucleic acid molecule also need not be full-length relative to either the primary transcription product or fully processed mRNA. Generally, higher homology can be used to compensate for the use of a shorter sequence. Furthermore, the antisense molecule need not have the same intron or exon pattern, and homology of non-coding segments will be equally effective. Antisense phosphorothioate oligodeoxynucleotides (PS-ODNs) is exemplary of an antisense molecule specific for the S1P2 receptor.

In another embodiment, the S1P2 receptor antagonist comprises an siRNA. RNA interference (“RNAi”) is a method of post-transcriptional gene regulation that is conserved throughout many eukaryotic organisms. RNAi is induced by short (i.e., less than 30 nucleotide) double stranded RNA (“dsRNA”) molecules which are present in the cell. These short dsRNA molecules, called “short interfering RNA” or “siRNA,” cause the destruction of messenger RNAs (“mRNAs”) which share sequence homology with the siRNA to within one nucleotide resolution. Without being held to theory, it is believed that the siRNA and the targeted mRNA bind to an “RNA-induced silencing complex” or “RISC”, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore effective for inhibiting expression of a target gene.

siRNA comprises short double-stranded RNA of about 17 nucleotides to about 29 nucleotides in length, specifically about 19 to about 25 nucleotides in length, that are targeted to the target mRNA, that is, the S1P2 receptor. The siRNA comprise a sense RNA strand and a complementary antisense RNA strand annealed together by standard Watson-Crick base-pairing interactions (“base-paired”). The sense strand comprises a nucleic acid sequence which is identical to a target sequence contained within the target mRNA.

The sense and antisense strands of siRNA comprise two complementary, single-stranded RNA molecules, or comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules.

One or both strands of the siRNA can also comprise a 3′ overhang. A “3′ overhang” refers to at least one unpaired nucleotide extending from the 3′-end of a duplexed RNA strand. In one embodiment, the siRNA comprises at least one 3′ overhang of 1 to about 6 nucleotides (which includes ribonucleotides or deoxynucleotides) in length, specifically of 1 to about 5 nucleotides in length, more specifically of 1 to about 4 nucleotides in length, and particularly specifically of about 2 to about 4 nucleotides in length. In the embodiment in which both strands of the siRNA molecule comprise a 3′ overhang, the length of the overhangs can be the same or different for each strand. In one embodiment, the 3′ overhang is present on both strands of the siRNA, and is 2 nucleotides in length. For example, each strand of the siRNA of the can comprise 3′ overhangs of dithymidylic acid (“FT”) or diuridylic acid (“uu”). In order to enhance the stability of the siRNA, the 3′ overhangs can also be stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′;-deoxythymidine significantly enhances the nuclease resistance of the 3′ overhang in tissue culture medium.

The siRNA is obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. The siRNA expressed from recombinant plasmids is isolated from cultured cell expression systems by standard techniques, or is expressed intracellularly at or near the area of neovascularization in vivo. The siRNA can also be expressed from recombinant viral vectors intracellularly at or near the area of neovascularization in vivo. The recombinant viral vectors comprise sequences encoding the siRNA and a promoter for expressing the siRNA sequences. Exemplary promoters include, for example, the U6 or H1 RNA pol III promoter sequences and the cytomegalovirus promoter.

One skilled in the art can readily determine an effective amount of the siRNA to be administered to a given subject, by taking into account factors such as the size and weight of the subject; the extent of the neovascularization or disease penetration; the age, health and sex of the subject; the route of administration; and whether the administration is regional or systemic. Generally, an effective amount of the siRNA comprises an intercellular concentration at or near the neovascularization site of about 1 nanomolar (nM) to about 100 nM, specifically about 2 nM to about 50 nM, more specifically about 2.5 nM to about 10 nM. It is contemplated that greater or lesser amounts of siRNA can be administered.

The inventors herein investigated the role of S1P signaling in retinal vascular development and abnormal angiogenesis in the ROP model. Although ceramide metabolism has been implicated in retinal photoreceptor function and endocytosis, as well as in diabetic retinopathy, the functional role of S1P in retinal development and pathology has not been addressed. This is particularly important as S1P is now accepted as an angiogenic factor and an inducer of vascular maturation. A recent report suggests that inhibitors of sphingosine kinase, the enzyme that generates the S1P ligand, reduced retinal vascular leakage in the rat diabetic retinopathy model. The role of S1P receptors in retinal vasculature has not been addressed previously. It was shown herein that the S1P₂ receptor is induced during ischemia-driven retinopathy, peaking at the growth phase of pathologic neovascularization. Immunohistochemistry experiments demonstrated that it is expressed in the growing vessels of INL and GCL, highlighting structures of vascular tufts. It is likely that either hypoxia per se or hypoxia-responsive regulators such as VEGF, Ang-2 could modulate S1P₂ receptor expression.

S1P levels were quantitated in retinal tissue with a highly specific HPLC method and only low levels were found (5-30 μmol/mg of protein) (data not shown). This result contrasts with the high S1P levels found in plasma (about 0.4-1 μM). Therefore, during retinal hypoxia, concomitant vascular permeability would likely result in S1P release in the interstitial milieu of the retina, which may activate the S1P₂ receptor on the vascular endothelial cells.

A major finding is that the S1P₂ receptor is essential for the pathologic angiogenesis of the retina. In P15 and P17, pathological intravitreal neovascularization was decreased by about 50% in S1p2^{-/-} retinas. S1p2^{-/-} retinas display enhanced intraretinal revascularization. Enhanced “physiological” revascularization in the S1p2^{-/-} retina was further confirmed by staining whole mount retinas for astrocytes (astroglia) and pericytes (mural cells), which were associated normally with endothelial cells. Thus, lack of S1P₂ shifted the phenotype of the ischemic retinal vasculature from “pathologic” to “normal”.

To further probe cellular mechanisms, endothelial cell proliferation was quantified; however, the same number of BrdU positive endothelial cells was observed in both WT and KO retinas at the beginning of pathogenesis (P14), suggesting that regulation of cell proliferation by S1P₂ is unlikely to be the primary mechanism involved. Detailed examination of the growing vessels at the beginning of neovascularization suggested that S1p2^{-/-} mice display increased number of endothelial cells with elongated processes (tip cells) that are oriented towards the avascular regions compared to S1p2^{+/+} mice. This alteration in normal vascular patterning may be disrupted by increased expression and signaling of S1P₂ in the endothelial cells, thus allowing misdirected angiogenesis in the vitreous chamber and concomitant reduced normal retina revascularization. It is possible that exaggerated S1P₂ signaling in endothelial cells could contribute to patterning defects. Without being held to theory, it is believed that retinal endothelial tip cell directionality, which is proposed to be regulated by signaling pathways such as VEGF and Notch, may be disrupted by aberrant S1P₂ signaling in the context of ROP. Such processes may also be influenced by inflammation.

Another key finding is that the S1P₂ receptor regulates inflammatory events in the ROP model. Wild-type retinas appear to be poorly perfused in areas of vascular tufts, whereas there are evident endothelial gaps and extravasation of the tracer into the abluminal space. In sharp contrast, at P17, S1p2 null retinas have improved blood flow and reduced leaky inflamed focal sites. This result is consistent with a recent study wherein it was shown that the S1P₂ receptor induced Rho- and PTEN-dependent paracellular permeability in endothelial cells and oxidant-induced lung vascular permeability. This increase in vascular permeability in the ischemic retinal vasculature is likely the key initiator of the inflammatory events. F4/80 positive myeloid cells were observed in the vascular tufts in WT animals whereas less inflammatory cells were associated with the retinal tissue of KO animals even at the very beginning of the pathogenesis.

Inflammatory mechanisms are thought to contribute to pathologic intravitreal angiogenesis. Inducible nitric oxide synthase (iNOS) inhibits angiogenesis in the avascular retina through the VEGFVEGR2 axis, thus leading to increased intravitreal angiogenesis. It has been shown that activated microglia contribute to enhanced revascularization by restoring “appropriate” gradient of angiogenic factors. The data shown herein suggest that the S1P₂ receptor-dependent inflammatory response may be important in the initiation and progression of abnormal ocular angiogenesis. Furthermore, abnormal intravitreal angiogenesis and normal retinal vascularization vessel may be inter-dependent. The data suggest that S1P signaling via the S1P2 receptor may alter the balance between these processes.

In order to gain insight into the molecular mechanisms by which the S1P2 receptor facilitates intravitreal neovascularization, the expression of pro-angiogenic and pro-inflammatory molecules was profiled. The pro-inflammatory molecule COX-2 is significantly reduced in KO retinas. This observation is in agreement with previous reports that identify COX-2 as a promoter of retina neovascularization, corneal neovascularization as well as tumor angiogenesis. Indeed, COX-2 specific inhibitors significantly reduced vascular tufts in the ROP model. Without being held to theory, it is believed that in the ischemic retina, the S1P₂ receptor induces COX-2, leading to increased inflammatory response and enhanced intravitreal neovascularization most likely through pro-angiogenic prostaglandin E₂ (PGE₂).

COX-2 inhibitor treatment, however, did not stimulate normal retinal vascularization, suggesting that additional targets regulated by S1P2 are involved. Without being held to theory, it is proposed that eNOS may be such a molecule. It was observed that at the early stage of pathological angiogenesis (P14), S1p2^{-/-} retinas have increased expression of eNOS protein in comparison with S1p2^{+/+} retinas. By performing in vitro experiments in endothelial cells, it was determined herein that the S1P₂ receptor directly downregulates eNOS protein expression. eNOS is a major source of NO, a potent vasodilator that facilitates proper blood flow and inhibits microvascular congestion. Interestingly, the observations reported herein are somewhat discordant to a previous publication wherein eNOS deficient mice develop reduced intravitreal neovascularization in oxygen-induced retinopathy. This may be due to the fact that eNOS deficient mice are less susceptible to hyperoxia-induced pruning of the vessels from P7 to P12, whereas increased eNOS expression was observed in S1p2^{-/-} hypoxic retinas at P14. In addition, the pharmacologic NOS inhibitor (L-NNA) that was used for these studies appears to be a competitive non-selective inhibitor of all three nitric oxide synthase isoforms (eNOS, nNOS and iNOS). It has been reported that eNOS mRNA stability is reduced under hypoxic conditions or upon thrombin stimulation and inflammation. Interestingly, Rho/Rho-associated kinase activation that is downstream of S1P₂/G_{12/13} receptor pathway is known to mediate hypoxia-dependent inhibition of eNOS expression in endothelial cells. Thus, it is hypothesized that in ischemic retinas S1P₂ receptor negatively regulates eNOS expression, possibly through Rho/Rho kinase pathway thus leading to retinal vascular congestion. Indeed, the data presented herein with the Rho kinase inhibitor supports this mechanism.

It has been shown herein for the first time that the S1P2 receptor pathway is an essential inducer of pathological neovascularization and inhibits hypoxia-triggered revascularization in the retina. Therapeutic compounds that specifically inhibit S1P₂ G-protein coupled receptors would inhibit pathologic angiogenesis while promoting “physiological” revascularization of the ischemic retina. Regulation of the plasticity of vascular phenotype by S1P₂ may also be useful in other ischemia-driven vascular diseases.

Disclosed herein are methods of treatment comprising administering to a subject an effective amount of an S1P₂ receptor antagonist. In one aspect, the agent is substantially purified (e.g., substantially free from substances that limit its effect or produce undesired side-effects). The subject is specifically an animal, e.g., such as cows, pigs, horses, chickens, cats, dogs, etc., and is more specifically a mammal, and most specifically a human.

The phrase “ophthalmically acceptable” with respect to a formulation, composition or ingredient such as an excipient means having no persistent effect that is substantially detrimental to the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

In one embodiment, the pharmaceutical compositions are administered to the area in need of treatment by topical administration. As used herein, topical administration refers to application to a localized area of the body or to the surface of a body part. Topical drug delivery is the most common treatment for diseases or disorders of the anterior segment of the eye, including, for example, corneal diseases, uveitis, and glaucoma. Topical delivery can be a safer and more convenient delivery method for patients, and can reduce the risk of many side effects observed in systemic treatment regimens. Topical administration of an angiogenesis inhibitor to the eye or cornea can be an effective treatment for treating neovascularization and/or inflammation. An exemplary method of administering the pharmaceutical compositions disclosed herein to the eye is by eye drops comprising an S1P₂ receptor antagonist.

In some embodiments, the pharmaceutical compositions are administered to the area in need of treatment by subconjunctival administration. One method of subconjunctival administration to the eye is by injectable formulations an S1P₂ receptor antagonist. Another preferred method of subconjunctival administration is by implantations comprising slow releasing an S1P₂ receptor antagonist.

Pharmaceutical compositions include a therapeutically effective amount of an active agent with a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Examples of suitable pharmaceutical carriers are described in “Remington's Pharmaceutical Sciences” by E.W. Martin. In one embodiment, the composition is formulated in accordance with routine procedures as a pharmaceutical composition adapted for topical administration to human beings. Such pharmaceutical compositions are liquid, gel, ointment, salve, slow release formulations or other formulations suitable for ophthalmic administration. The composition comprises an effective amount of an S1P₂ receptor antagonist and, optionally, at least one ophthalmically acceptable excipient, wherein the excipient is able to reduce a rate of removal of the composition from the eye by lacrimation, such that the composition has an effective residence time in the eye of about 2 hours to about 24 hours.

In various embodiments, compositions comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

In one embodiment, a liquid composition is aqueous. Alternatively, the composition can take form of an ointment. In one embodiment, the composition is an in situ gellable aqueous composition, more preferably an in situ gellable aqueous solution. Such a composition can comprise a gelling agent in a concentration effective to promote gelling upon contact with the eye or lacrimal fluid in the exterior of the eye. Suitable gelling agents non-restrictively include thermosetting polymers such as tetra-substituted ethylene diamine block copolymers of ethylene oxide and propylene oxide (e.g., poloxamine 1307); polycarbophil; and polysaccharides such as gellan, carrageenan (e.g., kappa-carrageenan and iota-carrageenan), chitosan and alginate gums. The phrase “in situ gellable” includes not only liquids of low viscosity that can form gels upon contact with the eye or with lacrimal fluid in the exterior of the eye, but also more viscous liquids such as semi-fluid and thixotropic gels that exhibit substantially increased viscosity or gel stiffness upon administration to the eye or area surrounding the eye.

Aqueous compositions can have ophthalmically compatible pH and osmolality. Specifically these compositions incorporate means to inhibit microbial growth, for example through preparation and packaging under sterile conditions and/or through inclusion of an antimicrobially effective amount of an ophthalmically acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof, phenoxyethanol; chlorophenoxyethanol; phenoxypropanol; chlorobutanol; chlorocresol; phenylethyl alcohol; disodium EDTA; and sorbic acid and salts thereof.

In one embodiment, the composition comprises an ophthalmic depot formulation comprising an active agent for subconjunctival administration. The ophthalmic depot formulation comprises microparticles of essentially pure active agent. The microparticles can be embedded in a biocompatible pharmaceutically acceptable polymer or a lipid encapsulating agent. The depot formulations may be adapted to release all of substantially all the active material over an extended period of time. The polymer or lipid matrix, if present, may be adapted to degrade sufficiently to be transported from the site of administration after release of all or substantially all of the active agent. The depot formulation can be liquid formulation, comprising a pharmaceutical acceptable polymer and a dissolved or dispersed active agent. Upon injection, the polymer forms a depot at the injections site, e.g. by gelifying or precipitating.

The composition can comprise a solid article suitable for insertion in a suitable location in the eye, such as between the eye and eyelid or in the conjunctival sac, where the article releases the active agent. Release from such an article is preferably to the cornea, either via lacrimal fluid that bathes the surface of the cornea, or directly to the cornea itself, with which the solid article is generally in intimate contact. Solid articles suitable for implantation in the eye in such fashion generally comprise polymers and can be bioerodible or non-bioerodible. Bioerodible polymers that can be used in preparation of ocular implants carrying an active agent include without restriction aliphatic polyesters such as polymers and copolymers of poly(glycolide), poly(lactide), poly(ε-caprolactone), poly(hydroxybutyrate) and poly(hydroxyvalerate), polyamino acids, polyorthoesters, polyanhydrides, aliphatic polycarbonates and polyether lactones. Illustrative of suitable non-bioerodible polymers are silicone elastomers.

In a further embodiment, one or more of the inhibitors, blockers, antagonists or regulators of the S1P2 receptor is administered to a mammalian subject in combination with a delivery system known to be effective for delivering agents for treatment of diseases and conditions of the eye. Such a delivery system is DuraSite®, available from InSite Vision, Inc. DuraSite® is a drug delivery vehicle that stabilizes small molecules in a polymeric mucoadhesive matrix. The topical ophthalmic solution can be described as a gel forming drop, which extends the residence time of the drug relative to conventional eye drops.

For oral administration, the pharmaceutical preparation can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Preparations for oral administration can be suitably formulated to give controlled release of the active compound.

For buccal administration, the compositions can take the form of tablets or lozenges formulated in conventional manner.

For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion via either intravenous, intraperitoneal or subcutaneous injection. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compositions can be formulated into creams, lotions, ointments or tinctures, e.g., containing conventional bases, such as hydrocarbons, petrolatum, lanolin, waxes, glycerin, or alcohol. The compositions can also be formulated in rectal compositions such as suppositories or retention enemas, e.g., containing conventional suppository bases such as cocoa butter or other glycerides.

In addition to the formulations described previously, the compositions can also be formulated as a depot preparation. Such long acting formulations can be administered by implantation (e.g., subcutaneously or intramuscularly) or by intramuscular injection. Thus, for example, the compositions can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

The amount of the S1P2 receptor antagonist that may be combined with pharmaceutically acceptable excipients to produce a single dosage form will vary depending upon the host treated and the particular mode of administration. The specific therapeutically effective amount for a particular patient will depend on a variety of factors including the activity of the specific compound employed, the age, body weight, general health, sex, diet, time of administration, route of administration, rate of excretion, drug combination, and the severity of the particular disease undergoing therapy. In some instances, dosage levels below the lower limit of the aforesaid range may be more than adequate, while in other cases still larger doses may be employed without causing any harmful side effects provided that such higher dose levels are first divided into several small doses for administration throughout the day. The concentrations of the compounds described herein found in therapeutic compositions will vary depending upon a number of factors, including the dosage of the drug to be administered, the chemical characteristics (e.g., hydrophobicity) of the compounds employed, and the route of administration. In general terms, the S1P2 receptor antagonist may be provided in an aqueous physiological buffer solution (for example, 1 cc) containing about 0.2% w/v compound for oral administration. The preferred dosage of drug to be administered is likely to depend on such variables as the type and extent of progression of the disease or disorder, the overall health status of the particular patient, the relative biological efficacy of the compound selected, and formulation of the compound excipient, and its route of administration, as well as other factors, including bioavailability, which is in turn influenced by several factors.

In various embodiments, the S1P₂ receptor antagonists may be administered in combination with one or more additional compounds or therapies or medical procedures. For example, suitable therapeutic agents for use in combination, either alternating or simultaneously, with the S1P₂ receptor antagonists, including topically administered immunosuppressive agents such as corticosteroids, dexamethasone, cyclosporin A, FK506, or anti-metabolic agents.

In one embodiment, a method of screening candidate molecules as potential inhibitors of an S1P2 receptor comprises contacting S1P2R-expressing retinal endothelial cells in culture with a candidate molecule, measuring the increase and/or induction of vascular or paracellular permeability in the retinal endothelial cells, determining if the candidate molecule is an inhibitor of an S1P2 receptor, and producing the molecule.

A candidate molecule comprises, but is not limited to, at least one of a lipid, nucleic acid, peptide, small organic or inorganic molecule, chemical compound, element, saccharide, isotope, carbohydrate, imaging agent, lipoprotein, glycoprotein, enzyme, analytical probe, and an antibody or fragment thereof, any combination of any of the foregoing, and any chemical modification or variant of any of the foregoing. In addition, a candidate molecule may optionally comprise a detectable label. Such labels include, but are not limited to, enzymatic labels, radioisotope or radioactive compounds or elements, fluorescent compounds or metals, chemiluminescent compounds and bioluminescent compounds. Well known methods may be used for attaching such a detectable label to a candidate molecule.

Methods useful for synthesizing candidate molecules such as lipids, nucleic acids, peptides, small organic or inorganic molecules, chemical compounds, saccharides, isotopes, carbohydrates, imaging agents, lipoproteins, glycoproteins, enzymes, analytical probes, antibodies, and antibody fragments are well known in the art. Such methods include the approach of synthesizing one such candidate molecule, such as a single defined peptide, one at a time, as well as combined synthesis of multiple candidate molecules in one or more containers. Such multiple candidate molecules may include one or more variants of a previously identified candidate molecule. Methods for combined synthesis of multiple candidate molecules are particularly useful in preparing combinatorial libraries, which may be used in screening techniques known in the art.

By way of example, multiple peptides and oligonucleotides may be simultaneously synthesized. Candidate molecules that are small peptides, up to about 50 amino acids in length, may be synthesized using standard solid-phase peptide synthesis procedures. For example, during synthesis, N-α-protected amino acids having protected side chains are added stepwise to a growing polypeptide chain linked by its C-terminal end to an insoluble polymeric support, e.g., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Many of the known methods useful in synthesizing compounds may be automated, or may otherwise be practiced on a commercial scale. As such, once a candidate molecule has been identified as having commercial potential, mass quantities of that molecule may easily be produced. Candidate molecules can be designed entirely de novo or may be based upon a pre-existing S1P2 receptor antagonist.

The invention is illustrated by the following non-limiting examples.

### EXAMPLES

### Materials and Methods

Animals. C57BL/6×129Sv mice with targeted disruption of the S1p2 gene were generated as previously reported. Mice were maintained on a mixed C57BL/6×129Sv genetic background and experiments on knockout (KO) mice were performed with appropriate littermate controls. All procedures involving mice were approved by the University of Connecticut Health Center Animal Care Committee. ROP was induced according to the following protocol. Briefly, pups (P7) with nursing mother were transferred into an air-tight incubator and were exposed to an atmosphere of 74±1% oxygen for 5 days. At P12, pups were returned to room air.

RNA isolation and RT-PCR analysis. RNA was extracted (RNeasy kit;Qiagen) from mouse retinas. First-strand cDNA was synthesized using random hexamers, murine leukemia virus reverse transcriptase and accompanying reagents (Invitrogen Corp.) for 1 hr at 37° C. Mouse RT-PCR primers shown in Table 1 were designed with Primer Express software (Applied Biosystems). Amplification and data analysis was performed in ABI Prism 7900HT Sequence Detection System (Applied Biosystems). mRNA levels were quantified and corrected for Cyclophilin A and expressed as fold induction over the corresponding control.

TABLE 1

Primer sequences for
quantitative real-time RT-PCR

| | |
|---|---|
| Gene | Sequence of primers |

| | |
|---|---|
| mSlp1 | F: ATGGTGTCCACTAGCATCCC |
| | SEQ ID NO: 1 |
| | R: CGATGTTCAACTTGCCTGTGTAG |
| | SEQ ID NO: 2 |
| mSlp2 | F: ATGGGCGGCTTATACTCAGAG |
| | SEQ ID NO: 3 |
| | R: GCGCAGCACAAGATGATGAT |
| | SEQ ID NO: 4 |
| mSlp3 | F: GCCTAGCGGGAGAGAAACCT |
| | SEQ ID NO: 5 |
| | R: CCGACTGCGGGAAGAGTGT |
| | SEQ ID NO: 6 |
| mAng2 | F: TCAACAGCTTGCTGACCATGAT |
| | SEQ ID NO: 7 |
| | R: GGTTTGCTCTTCTTTACGGATAGCAGC |
| | SEQ ID NO: 8 |
| MVEGF | F: CACGACAGAAGGAGAGCAGAAGT |
| | SEQ ID NO: 9 |
| | R: TTCGCTGGTAGACATCCATGAA |
| | SEQ ID NO: 10 |
| mFlt1 | F: GAGGAGGATGAGGGTGTCTATAGGT |
| | SEQ ID NO: 11 |
| | R: GTGATCAGCTCCAGGTTTGACTT |
| | SEQ ID NO: 12 |
| mTNF-a | F: GGGCCACCACGCTCTTCTGTCT |
| | SEQ ID NO: 13 |
| | R: GCCACTCCAGCTGCTCCTCCAC |
| | SEQ ID NO: 14 |
| MiNOS | F: TGGCCACCTTGTTCAGCTACG |
| | SEQ ID NO: 15 |
| | R: GCCAAGGCCAAACACAGCATAC |
| | SEQ ID NO: 16 |
| mCOX-2 | F: GTACCCGGACTGGATTCTATGG |
| | SEQ ID NO: 17 |
| | R: GGGTGGGCTTCAGCAGTAATT |
| | SEQ ID NO: 18 |
| MCyclophilin | F: ATGGCAAATGCTGGACCAAA |
| | SEQ ID NO: 19 |
| | R: TGCCATCCAGCCATTCAGT |
| | SEQ ID NO: 20 |
| MGAPDH | F: CAACTACATGGTCTACATGTTCCAGT |
| | SEQ ID NO: 21 |
| | R: TGACCCGTTTGGCTCCA |
| | SEQ ID NO: 22 |

The S1P2R gene from human (Accession Number NM_{—}004230; SEQ ID NO: 23) is given below:

```
      1 cggccgccct ggggacgcag acgccaaggc ccctccggcc agggccggga gccgggccgg
    61 cctagccagt tctgaaagcc ccatggcccc agcaggcctc tgagccccac catgggcagc
  121 ttgtactcgg agtacctgaa ccccaacaag gtccaggaac actataatta taccaaggag
  181 acgctggaaa cgcaggagac gacctcccgc caggtggcct cggccttcat cgtcatcctc
  241 tgttgcgcca ttgtggtgga aaaccttctg gtgctcattg cggtggcccg aaacagcaag
  301 ttccactcgg caatgtacct gtttctgggc aacctggccg cctccgatct actggcaggc
  361 gtggccttcg tagccaatac cttgctctct ggctctgtca cgctgaggct gacgcctgtg
  421 cagtggtttg cccgggaggg ctctgccttc atcacgctct cggcctctgt cttcagcctc
  481 ctggccatcg ccattgagcg ccacgtggcc attgccaagg tcaagctgta tggcagcgac
  541 aagagctgcc gcatgcttct gctcatcggg gcctcgtggc tcatctcgct ggtcctcggt
  601 ggcctgccca tccttggctg gaactgcctg ggccacctcg aggcctgctc cactgtcctg
  661 cctctctacg ccaagcatta tgtgctgtgc gtggtgacca tcttctccat catcctgttg
  721 gccatcgtgg ccctgtacgt gcgcatctac tgcgtggtcc gctcaagcca cgctgacatg
  781 gccgccccgc agacgctagc cctgctcaag acggtcacca tcgtgctagg cgtctttatc
  841 gtctgctggc tgcccgcctt cagcatcctc cttctggact atgcctgtcc cgtccactcc
  901 tgcccgatcc tctacaaagc ccactacttt ttcgccgtct ccaccctgaa ttccctgctc
  961 aaccccgtca tctacacgtg gcgcagccgg gacctgcggc gggaggtgct tcggccgctg
1021 cagtgctgga ggccgggggt gggggtgcaa ggacggaggc ggggcgggac cccgggccac
1081 cacctcctgc cactccgcag ctccagctcc ctggagaggg gcatgcacat gcccacgtca
1141 cccacgtttc tggagggcaa cacggtggtc tgagggtggg ggtggaccaa caaccaggcc
1201 agggcagagg ggttcatgga gaggccactg ggtgacccca gatagagact tggggctact
1261 gagccagatg cccccgcccc acagacctgg gtgatgttgc aaatatttca cacctggaaa
1321 ggccagataa ggcactgact agtcacatag cagtgttgca gtgcggtcct gagggccagt
1381 ccagtggcta gtgtgacccc tttagaactg gatcctgggg aggccagggc aggggacctg
1441 tgaagagcca gggtgagggc aggcagcatt taaggggagc tcagggcagg agcactttac
1501 cacctggtac aaaggatttt tttttttttt tgagacggaa tcttgcactg ctgcccaggc
1561 tggagtgcag tggcgtgatc tcggctcacc gcaagctccg cctcctgggt tcatgtcgtt
1621 ctcctgcctc agcctcccaa gtagctggga ctataggcgc ctgccaccac acctggctaa
1681 ttttttgtac ctttagtaga gatggggttt caccgtgtta gccaggatgg tcttgatctc
1741 ctgacctcgt gatccgcccg cctcggcctc ccaaagtgct gggattacag gcgtgagcca
1801 ccgtgcccgg cttttttttt tttttttttt tttttttttt ttttttttga gatgaagtct
1861 cgctctgttg cccaggctgg agagtgcagt ggtacggtct cagctcactg caacctccac
1921 ctcccaggtt caagcgattc tccagcctga gcctcctgag tagctgggat tacaggtgcc
1981 taccaccacg cccaggtaat tttttttttt tttgtatttt tagtagagac ggggtttcac
2041 catgttggcc aggctggtct cgaactcctg acctcatgat ccgcccgtgt tggcctccca
2101 aagtgtggga ttacaggcgt aagccacctc acctggcggt acaaagaatt tctgcatttt
2161 cttccctggc ccctagtcct gcaccgattt ctccttttcg aatgtattcc tcctgccacc
2221 ttctctgggc aacttcgtgc gactacagaa ccactgtcct gaggagctag aggcctcctc
2281 tctgaccatc cagagcccaa atccacagct tccccaaatt tcatcagctg ccacttgacg
2341 acttctcccc gtctctctga ggcccggaaa ccacggctgg aggtggggag gggatggcgg
2401 ctgaggtcca ttcctcattc tcagacctca ttgctcagtt gcactatttg gggcacagaa
2461 taatcaccaa aagtgagaaa aacgagtttg ggtggctggg gaggactttg ggactcttga
2521 tgcaaggcgc aacttgagaa aattctgggt gtgatatttg cacagacacc ctcctttcaa
2581 aaacagccac cccccaagct attctcagct ccacacctgc agccccagct aaggtaccag
2641 gtctcctgag caaggcagag agaagccttg agccttctct gtgtcttctt tcaagaaccc
2701 cgctgtgtct tctttcaaga tttttttttt gagacagttt caagattttt gttttgtttt
2761 tgagatggag tctcactgtg tcacccaggc tgaggtggca gtggttcaat ctccgttcac
2821 tgccacctcc acctcccggg ttcaagcgat tctcctgctt cagcctctcg agtagctggg
2881 actacaggca cctgccacca tgtctggcta atttttgtat ttttagtaga gacagggttt
2941 cactacgttg gccaggctgg tctcaaactc ctgacctcaa gtgatccgcc cgcctcggcc
3001 tccccaattg ctgggattac aggcgtgagc cactgtgccc ggccttcttc tttcaagtta
3061 tatagaatgg agcatggggg tggcagtggc tagggacatt tcctggggac actctcccct
3121 aaccccccag aaggacttca caaaaacctg tggataatgg aagggatgtt acggtacaaa
3181 cgtatattta tgtgtgtgtg tgtgtatgtg tgtgcgcgcg cgcgtgtgca cataggcgtg
3241 atgtctgtga ccctcctctc ctcgtcacat ttcccccaga atgaatgctg tcctgtctgc
3301 tcatgtttgt gttgaagctg ccaaagtcgg ggagctctgg tcctgcccag acccctttgg
3361 aattgctggc ccatcctccc actggagagc tggggtgcag ctcaccttgg ggaaggaaac
3421 ctcatgcctc agagtaattt cttgtgaatg caaagcctgg gggagcgggt ctttgggggg
3481 caaggagcca gtcaggggct tgtttcccct catagagctc cccagacgtg cctccgcaat
3541 gcctgaaacc cagacctagg ctaataaacg gttcaatttc tgttaaaaa
```

### Histology and Immunohistochemistry.

For intravitreal neovascularization, twelve sections (6 μm thick, 30 μm apart) from each eyeball were periodic-Schiff (PAS) and Hematoxylin stained (PAS kit, Sigma). Vascular cell nuclei growing beyond INL were counted. For S1P₂ staining, heat epitope retrieval of tissue sections was performed in 10 mM Citrate buffer (pH 6.0). For F4/80 and COX-2 staining, tissue sections were pretreated with pronase (Sigma) for 5 minutes. Sections were stained with primary antibody overnight at 4° C.: rabbit polyclonal anti-S1P₂ receptor (1:200), mouse anti-F4/80 (1:100, BD Pharmigen), rabbit anti-COX-2 (1:800, Cayman Chemical). Primary antibody detection was performed with Vector ABC kit (Vector Laboratories). Counterstaining with Methyl Green or Mayer's Hematoxylin.

### Retina Whole Mount Preparation, Quantification of Avascular Area and Immunoflurescence.

Eyes were enucleated and fixed in 4% PFA for 15 minutes. Retinas were dissected out and post-fixed for 15 minutes. To visualize endothelium, retinas were stained with Alexa-594 conjugated GS Lectin (20 ug/ml, Molecular Probes, 1:200 in blocking buffer). Primary antibodies: FITC-conjugated mouse anti-α-smooth muscle actin (1:100, Sigma), rabbit anti-GFAP (1:200, DAKO), rabbit anti-NG2 (1:200, Chemicon), mouse anti-BrdU (1:200, Chemicon). Secondary antibodies: Alexa-Fluor 488-conjugated goat anti-rabbit antibody (1:200, Molecular Probes). Retinas were visualized by using LSM-510 Confocal Microscope. Avascular and total retina areas were quantified with Image J software. For FITC-RCA I (50 μl, 2 mg/ml, Vector Lab.) perfusion, mice were anesthetized with Avertin (Sigma), injected with RCA I in the left ventricle which was allowed to circulate for 2 minutes. Unbound lectin was removed with 1% BSA-PBS perfusion for 1 min followed with 4% PFA-PBS fixation for 5 minutes. Eyes were enucleated, postfixed and stained as previously described. For tip cell quantification, sprouts were counted in four different fields of the retinal mid-periphery and the mean number of tip cells per retina was calculated. For BrdU cell quantification, Image-Pro Plus image analysis software was used to count fluorescent pixels per total retinal area. Luciferase activity experiments.

EOMA cultures (3×10⁵ cells/well) on 6-well plate were grown one day before the transfection. To measure transfection efficiency, cells were co-transfected with pCMV-βgal. For each well, 0.3 μg of the luciferase reporter vector phPES2(−1432/+59), 0.3 μg of the gene of interest (pcDNA3.1-S1p2 or pcDNA3.1-S1p1) and 25 ng of pCMV-βgal mixed with Lipofectin 2000 (Invitrogen) were introduced into the cells as described by the manufacturer. 24 hours after transfection, cells were treated with PMA (100 nm) for 5 hours, if necessary. Cells were harvested and luciferase and β-galactosidase activity were determined with Luciferase assay system (Promega) and Western-light and western-star system (Applied Biosystems) respectively. The amount of plasmid DNA was made constant by adding pcDNA 3.1 and luciferase activity was normalized to μg of protein content.

### Western Blot.

Retinas or cells were solubilized in 2×SDS-sample buffer (20 mM DTT, 6% SDS, 0.25M Tris pH 6.8, 10% Glycerol, bromophenyl blue, protease inhibitors, 1 mM sodium orthovanadate and 1 mM NaF), sonicated, boiled and separated by SDS-PAGE gel electrophoresis. Membranes were incubated with the following antibodies: anti-actin (Sigma), anti-eNOS (BD Pharmingen), anti-COX-2 (Cayman) and anti-V5 (Invitrogen). Cells were treated overnight with 10 μM Y-27632 (Calbiochem). Immunoreactive bands density was quantified with IQMac version 1.2 software.

### Adenoviral Vector Construction and Production.

cDNA encoding S1P2-V5 tag was subcloned into the pShuttle-cytomegalovirus vector that was used to produce recombinant adenovirus by using bacteria-AdEasy vector system (AdEasy kit, Quantum Biotechnologies) as described by the manufacturer.

### Statistics.

Statistical differences were assessed using the 2-tailed Student's t Test. P values less than 0.05 were considered significant.

### Example 1

### S1P₂ Receptor Expression in the Course of Ischemia-Induced Pathologic Retinal Angiogenesis

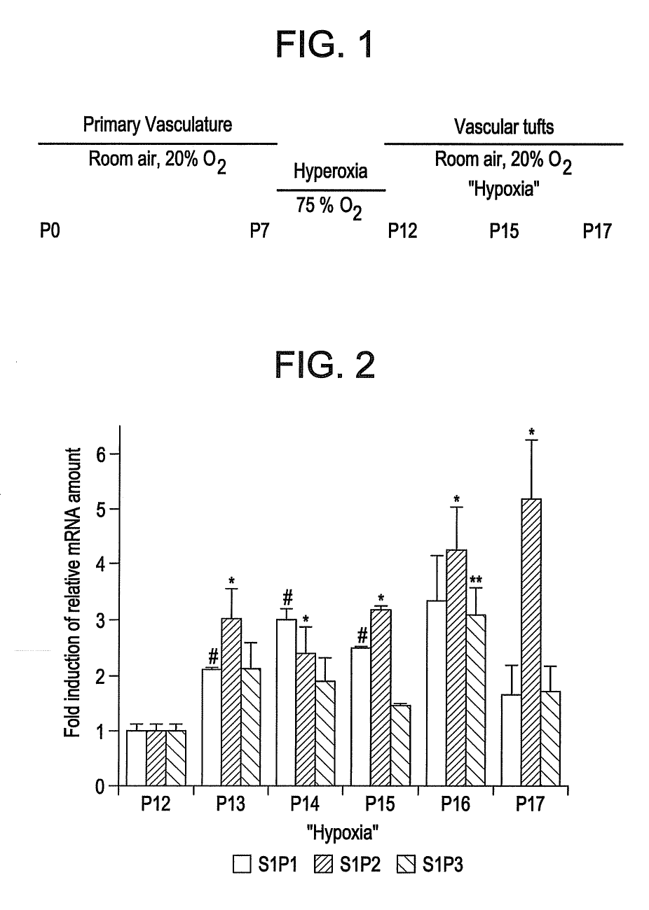

Ocular neovascularization, which leads to pathologic vessel growth, is the primary cause of severe eye diseases such as diabetic retinopathy, age-related macular degeneration and retinopathy of prematurity (ROP). In order to evaluate whether S1P receptors play a role in retina neovascularization, the expression of S1P receptors in a mouse model of retinal ischemia was investigated. After pups and their nursing mothers have been exposed to 75% oxygen-hyperoxia for 5 days (P7 to P12), the capillary network of the central retina regresses (vascular obliteration). At P12, pups and their nursing mothers were returned back to room air (“Hypoxia”). Resultant retinal ischemia initiates rapid vessel growth; however, pathologic angiogenesis occurs in the vitreous, reaching a maximum at P17 (FIG. 1).

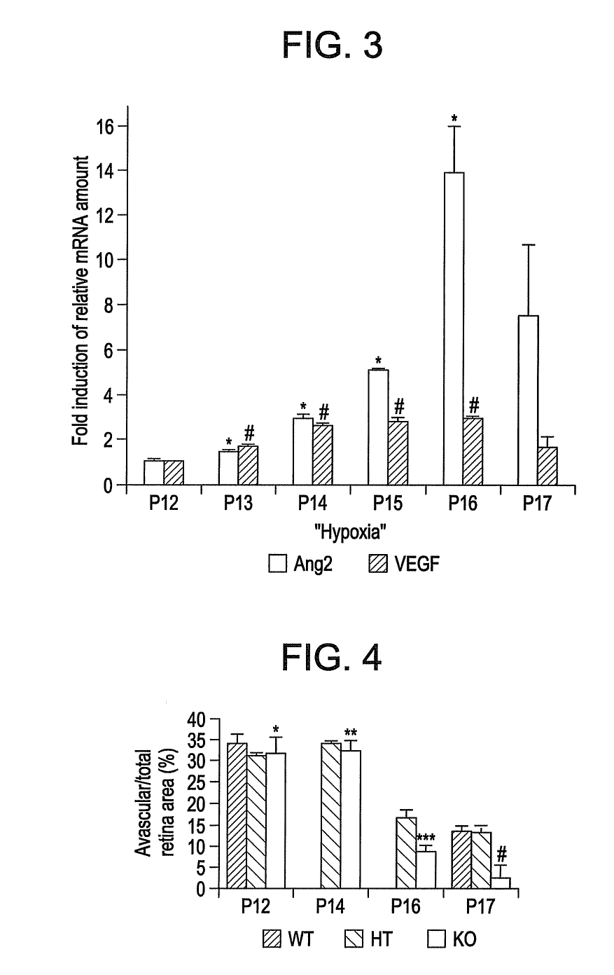

The expression of ubiquitously expressed S1P receptors, namely S1P₁, S1P₂ and S1P₃ was measured by quantitative real time RT-PCR (q-RT-PCR) assay (FIG. 2). Expression of all three receptors was detected at P12 before the onset of relative hypoxia. Interestingly, at P13 (24 hrs of relative “hypoxia”), the S1P₂ receptor mRNA level was increased 3-fold (P<0.035; n=3). The receptor expression increased further to 5-fold at P17 (5 days of relative “hypoxia”, P<0.035; n=3,) at the growth phase of pathologic angiogenesis. However, mRNA levels of S1P₁ and S1P₃ receptors increased modestly during the course of relative hypoxia and returned at baseline levels by P17 (FIG. 2). As expected, ischemia resulted in enhanced retinal expression of VEGF mRNA by more than 2.5-fold (P<0.0015; n=3, P16) and Ang-2 expression by 14-fold (P<0.015; n=3, P16), which is consistent with previous reports that describe VEGF and Ang-2 as hypoxia-induced regulators of retinal angiogenesis (FIG. 3). In contrast, during the course of normal retina development (normoxia), S1P₂ receptor expression sharply declined during the first week of vascular development (P5 to P10) and remained at low levels (P15, P28) (data not shown).

Prompted by the observation that S1P₂ mRNA expression is significantly increased during the course of relative hypoxia, the cells that express this receptor in the retina were localized. An S1P₂ antibody detected an appropriately sized (approximately 40 Kd) molecule in Western Blot analysis of protein extracts of VSMCs and mouse embryonic fibroblasts (MEFs) that endogenously express S1P₂ receptor as well as of human embryonic kidney 293 cells (HEK293) transfected with the S1P₂ receptor. (data not shown). These observations indicate that the antibody is specific in the detection of the S1P₂ receptor. The S1P₂ receptor was detected by immunohistochemistry in retinal cross sections around the optic nerve area at P17. S1P₂ staining exhibited a strong signal of vessel-like distribution in the ganglion cell layer and in the inner nuclear layer (INL, arrowheads) of hypoxic retinas. However, there was no immunoreactivity in the avascular outer nuclear layer (ONL) (data not shown). At higher magnification, it was evident that the S1P₂ receptor is expressed in endothelial cells of INL as well as in the primary vasculature of GCL, where S1P₂ expression highlights vascular tuft (VT) like structures that abnormally sprout at the interface between vitreous and retina (data not shown). These observations suggest that the S1P₂ receptor is significantly induced in ischemic retinal endothelium and underscore the possibility that its signaling in the endothelium is important in hypoxia-driven neovascularization.

### Example 2

### Enhanced Intraretinal Revascularization in S1p2^{-/-} Mouse Retina

Next, the phenotypes of S1p2^{+/+} and S1p2^{-/-} retinal whole mounts stained en face with Griffonia simplicifolia lectin (GS-lectin), at the peak of neovascularization (P17) (data not shown), were examined. Interestingly, S1p2^{-/-} retinas developed enhanced intraretinal revascularization, whereas the S1p2^{+/+} littermates showed increased avascular areas and formation of pathologic neovascular tufts, which is the expected phenotype of the ROP model. To further study the role of the S1P₂ receptor in intraretinal revascularization, GS-lectin stained retinal whole mounts were imaged at different stages during the course of the ROP model. In particular, avascular areas as percentage of total retinal area were measured in S1p2^{+/+}, S1p2^{+/+} and S1p2^{-/-} mice. At P12, vascular obliteration occupied approximately 35% of the total retina surface in S1p2^{+/+} (34.2±2.14%) as well as in S1p2^{+/-} (31.2±0.9%) and S1p2^{-/-} littermates (31.9±3.6%, P=0.37; FIG. 4). At P14 (2 days of hypoxia), no significant difference was observed in the avascular area of S1p2′ (34.19±0.74%) or S1p2^{-/-} mice (32.49±2.4%; P=0.1) (FIG. 4). Extensive revascularization in S1p2+/−retinas was evident at P 16 (4 days of hypoxia, data not shown); S1p2^{-/-} retinas showed significant decrease of capillary-free area (16.6±1.9% vs. 8.6±1.7%; P<0.001, FIG. 4). The difference was most prominent at P17 (13.5±1.3% vs. 2.6±2.9% of the total area; P<0.0001; FIG. 4). In the wild-type animals, the vessels are tortuous and dilated with evident abnormal vascular growth between vascularized periphery and capillary-free central area, peaking around the optic disc area (data not shown), indicating that S1p2^{-/-} retinas display normal vascular patterning and development during hypoxic insult.

Furthermore, detailed examination of frozen retinal cross sections during normal development (normoxia) revealed that developing vessels (GS-lectin staining) of S1p2^{-/-} retinas (P6) spread finely towards the periphery of the retina and are able to form deeper capillary networks (P12 and P20) similarly to their WT littermates (data not shown). In addition, vessels of S1p2^{-/-} normoxic retinas appear to follow the well defined astrocytic plexus while S1p2^{-/-} endothelial cells (tip cells) at the leading edge of the growing vessels display long filopodia similar to the WT counterparts (data not shown). Moreover, there was no obvious difference between the WT and KO counterparts in terms of vascular maturation and arteriovenous pattern formation as evidenced by α-SMA staining of whole mount retinas (data not shown). These observations indicate that S1P₂ receptor is induced and modulates retina vascular development under hypoxic conditions whereas it is dispensable for vascular development during the course of normal retina development.

### Example 3

### Abnormal Angiogenesis is Attenuated in the Intravitreal Region of S1p2^{-/-} -Mice

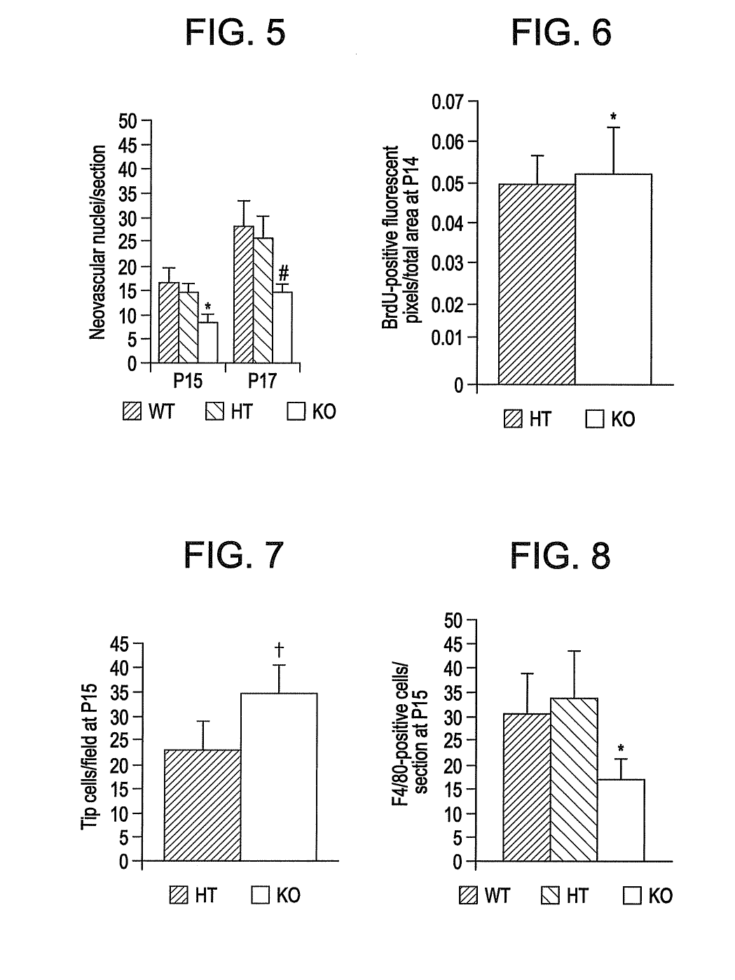

Intravitreal angiogenesis was determined by counting the nuclei of growing vessels that extend beyond the interface between the retina and vitreous (Inner limiting membrame, ILM) of periodic acid-Schiff (PAS)— and hematoxylin-stained serial cross sections (data not shown). S1p2^{+/+} and S1p2^{-/-} mice maintained in normoxia did not show intravitreal angiogenesis (data not shown). At P15, when pathological tufts start developing, the mean number of nuclei counted for S1p2^{+/+} and S1p2^{+/-} retinas was 22.5±3.7 and 19.58±2.43, respectively. The mean number of neovascular nuclei for S1p2^{-/-} retinas was 11.27±2.16 (P<0.0025; FIG. 5). At P17, when intravitreal neovascularization reached a maximum, the mean number of nuclei counted for S1p2^{+/+} and S1p2^{+/-} retinas that form vascular tufts (VT) was 37.6±7.03 and 34.528±6.2, respectively (FIG. 5). In sharp contrast, the mean number of neovascular nuclei of S1p2^{-/-} retinas was reduced by approximately 50% (19.62±2.2, P<0.001; FIG. 5). These data suggest that animals that lack S1p2 receptor display greatly reduced pathological intravitreal neovascularization starting at the early stages of the disease.

### Example 4

### Ischemic S1p2^{-/-} Mouse Retinas Display Normal Vascular Morphology

In order to characterize the enhanced intra-retinal revascularization in more detail, GS-lectin stained S1p2^{+/+} and S1p2^{-/-} whole mount retinas were imaged at P17, in the mid-peripheral region. Ischemic S1p2^{-/-} retinas exhibit nearly complete and well-defined architecture of the two additional capillary networks in inner plexiform (IPL) and outer plexiform (OPL) layers besides the primary vasculature of nerve fiber layer (NFL), whereas S1p2^{+/+} retinas form poorly organized capillary network in the OPL (data not shown). In addition, S1p2^{-/-} mice similarly to mice maintained in normoxia (data not shown) display normal, almost fully recovered intra-retinal vasculature in close association with surrounding long astrocytic processes (data not shown). In S1p2^{+/+} retinas, astrocytes (GFAP positive cells) cover the retina surface but they are not fully connected with abnormally growing vessels in the vascular tuft areas (data not shown). Furthermore, in S1p2^{+/+} retinas, although pericyte (NG2 positive cells) staining is apparent, pericyte coverage of endothelial cells in vascular tuft areas appears reduced (data not shown). In S1p2^{-/-} retinas, ensheathing pericytes that were engaged around vessels were observed, which is similar to normoxic retinas at P17 (data not shown). These observations indicate that at P17 (peak of neovascularization), S1p2^{-/-} retinas display normal formation of the primary as well as the deeper capillary retinal networks and increased normalization of the vasculature.

### Example 5

### S1P2 Receptor Modulates Vascular Patterning But not Proliferation in Ischemic Mouse Retina

Cellular events regulated by the S1P2 receptor we then examined. To study the proliferation of retinal vessels, BrdU incorporation assays were performed. BrdU positivity was most pronounced in endothelial cells (GS-lectin positive cells, data not shown); however a minor fraction of GFAP positive cells (astrocytes) also incorportated BrdU (data not shown). At P14 (very early stage of pathological neovascularization), both S1p2^{+/-} and S1p2^{-/-} retinas exhibited similar distribution of BrdU-positive endothelial cells in the primary vasculature (data not shown). Quantification of BrdU positive cell number was similar in S1p2^{+/-} vs. S1p2^{-/-} retinas at P14 (FIG. 6). At P17, in S1p2^{+/+} retinas, proliferating cells are mostly located in the vascular tuft areas that protrude towards the vitreous (data not shown). In contrast, mitogenic endothelial cells of S1p2^{-/-} retinas were distributed evenly in the well-formed vascular network of the central retina (data not shown). These data suggest that the intrinsic proliferation of endothelial cells is not modulated by S1P₂ signaling.

The next studies were focused on vascular patterning as a possible mechanism by which S1P₂ regulates the ROP phenotype. At P15, S1p2^{-/-} retinas contained new vascular sprouts directed into the central avascular region of the retina, whereas S1p2^{+/+} retinas displayed intravitreal neovascular tufts (data not shown). Interestingly, at P15, the mean number of tip cells that are directed into the avascular retina was 34.34±6.3 in S1p2^{-/-} retinas whereas it was only 23.12±5.73 (P<0.0025, FIG. 7) for the S1p2^{+/+}. GS-lectin stained S1p2^{+/+} and S1p2^{-/—} whole mount retinas were imaged at P17 when maximum revascularization of NFL was observed (data not shown). At higher magnification in S1p2^{+/+} retinas, growing vessels formed round abnormal buds that are oriented towards the vitreous (data not shown). In sharp contrast, in analogous areas of S1p2^{-/-} retinas, long tip cells were seen at the edge of newly growing vessels that are directed towards the avascular retina (data not shown). These data are consistent with the notion that S1P₂ receptor modulates the directionality and patterning of endothelial cells in the context of pathologic angiogenesis.

### Example 6

### S1P₂ Receptor Promotes Hypoxia-Driven Inflammatory Response in Mouse Retina

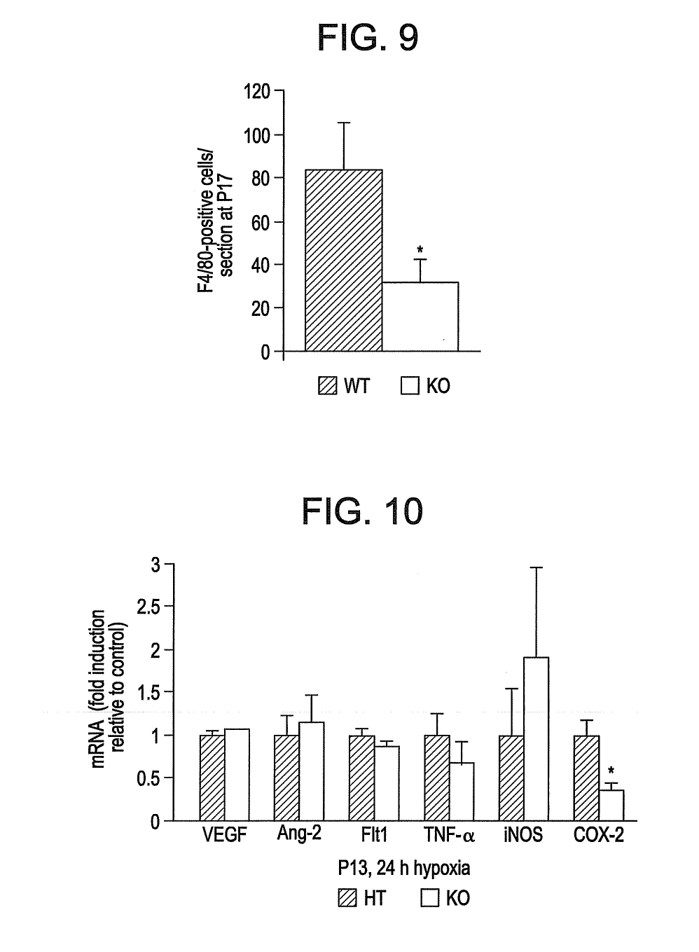

Pathologic angiogenesis in the mouse model of ischemia driven retinopathy is known to be regulated by hypoxia-mediated expression of angiogenic factors such as VEGF, Ang-2 and iNOS. However, recent reports suggest that inflammation may play a crucial role during the progression of ectopic neovascularization and vascular tuft formation. To test whether the inflammatory response is modulated, inflammatory cells of the myeloid lineage were probed in retinal cross sections, by macrophage-specific F4/80 immunostaining (data not shown). In P15 WT or HT retinas, the number of macrophages present in the vascular tuft area is 30.57±8.23 and 33.91±9.5, respectively (FIG. 8). In P15 KO retinas, the number of inflammatory cells is significantly reduced to 16.85±4.33 (P<0.02; FIG. 8). In P17 WT retinas, increased number of macrophages was present in the vascular tuft area, in the vicinity of abnormally growing blood vessels. Moreover, macrophages with long processes were localized between GCL and INL, representing inflammatory cells that infiltrate the tissue upon vascular damage (83.76±20.93 cells; FIG. 9). In sharp contrast, KO littermates display markedly reduced number of macrophages between GCL and INL in close association with vessels (31.81±9.88 cells, P<0.02; FIG. 9). These experiments suggest that the S1P₂ receptor is a critical mediator in the inflammatory response of the ischemic retina.

To further investigate the involvement of inflammation, FITC-conjugated Ricinus communis agglutinin I (RCA I) tracer was injected into the left ventricle of S1p2^{+/+} and S1p2^{-/-} M mice (P17). RCA I has been reported to label focal sites of plasma leakage and endothelial gaps of inflamed areas. Then, retinas were dissected and stained en face with Alexa 594-conjugated GS lectin to image the total retinal vasculature. In S1p2^{+/+} whole mount retinas, GS lectin staining highlights the whole vasculature whereas areas of vascular tufts were only partially perfused by the RCA I tracer (data not shown). However, in S1p2^{-/-} mice, RCA I tracer staining pattern was very uniform and co-incided with the GS lectin staining (data not shown). This suggested that perfusion of retinal vasculature is efficient in S1p2^{-/-} retina. In addition, in S1p2^{+/+} retinas, areas with significant RCA I extravasation into the basement membrane and abluminal space were observed, whereas few leaky gaps were evident in S1p2^{-/-} vessels. These data suggest that reduced vascular injury and improved blood flow of the retinal vasculature is characteristic of the S1p2^{-/-} mice at P17.

### Example 7

### The Proinflammatory Enzyme Cox-2 is Positively Regulated by S1P₂ Receptor

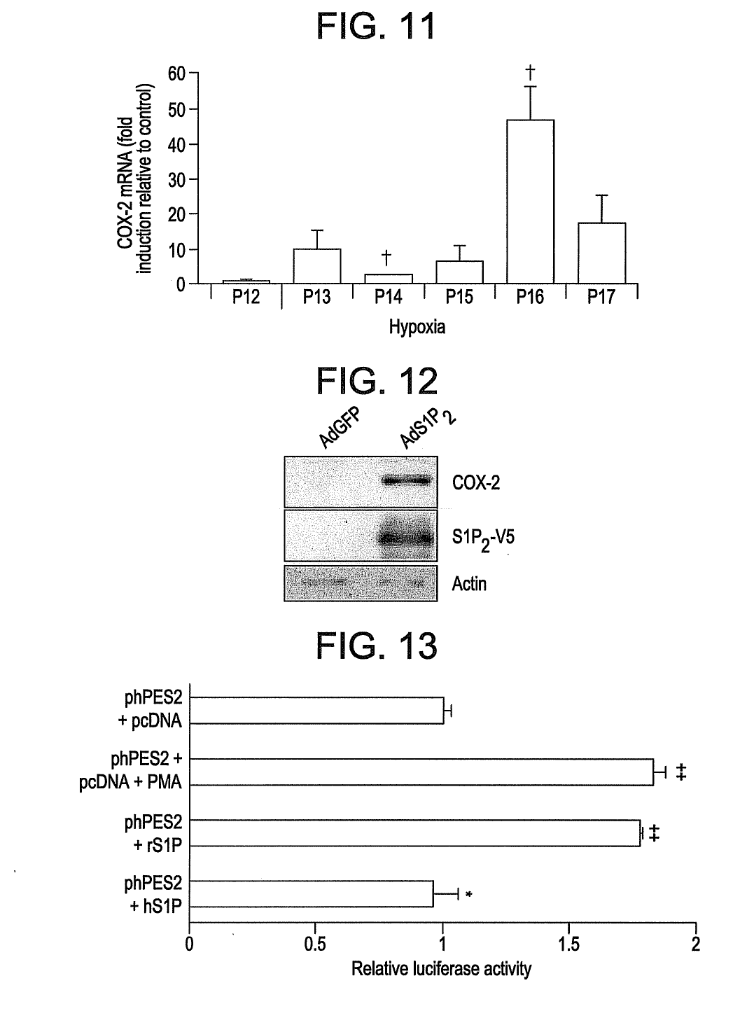

To elucidate the molecular mechanisms involved in the regulation of retinal vascularization and intravitreal angiogenesis by the S1P₂ receptor, mRNA expression of pro-angiogenic and pro-inflammatory mediators was studied at the very early onset of hypoxia (P13, 24 hours hypoxia), before the initiation of pathologic neovascularization. Expression of VEGF, Ang-2 and Flt1 (pro-angiogenic mediators) as well as TNFα and iNOS (pro-inflammatory mediators) was similar in S1p2^{+/-} and S1p2^{-/-} littermates (FIG. 10). However, COX-2 expression was significantly decreased by approximately 64% in the S1p2^{-/-} retinas compared to S1p2^{+/-} counterparts. In addition, in WT retinas, COX-2 mRNA expression is highly induced during the course of hypoxia, peaking at P16 (4 days of hypoxia) (FIG. 11). These experiments suggest that pro-inflammatory mediator COX-2 could be a target of S1P₂ receptor pathway, during the hypoxia driven inflammatory response in the mouse retina.

To gain further insights into the cellular mechanism of COX-2 regulation by S1P₂ receptor in hypoxic retina, immunohistochemistry was performed in retina cross sections to localize COX-2. Interestingly, S1p2^{+/+} retinas display strong COX-2 expression in the retinal nerve cells of INL and GCL. Enhanced COX-2 staining was observed in the endothelial cells of INL while COX-2 was predominantly expressed in the growing abnormal vessels of GCL, similarly to S1P₂ receptor tissue expression pattern (data not shown). In S1p2^{-/-} retinas, COX-2 was still detected in nerve cells of INL and GCL but its expression was significantly reduced in vessels of INL and mostly GCL (data not shown). These in vivo experiments suggest that in mouse ischemic retina, S1P₂ receptor function induces COX-2 expression in vascular endothelial cells.

When the S1P₂ receptor was expressed in HUVECs by adenoviral transduction, COX-2 protein expression was significantly increased relatively to control Ad-GFP transduced cells (FIG. 12). In addition, it was tested if S1P₂ receptor can induce the transcription of the COX-2 gene. For this experiment, the human COX-2 promoter (phPES2) driven reporter activity was measured in transfected endothelial cells. As expected, the promoter activity of phPES2 that contains the 5′-flanking region (−1432/+59) of the human PTGS2 (COX-2) gene was induced 1.83-fold by PMA in mouse hemangioendothelioma cells (EOMA). The promoter activity was induced 1.81-fold when S1p2 receptor was transfected while there was no induction observed in cells transfected with the S1p1 receptor (FIG. 13). Thus, these data suggest that the S1P₂ receptor induces the proinflammatory gene COX-2 at the transcriptional level.

### Example 8

### eNOS Expression is Negatively Regulated by the S1P₂ Receptor

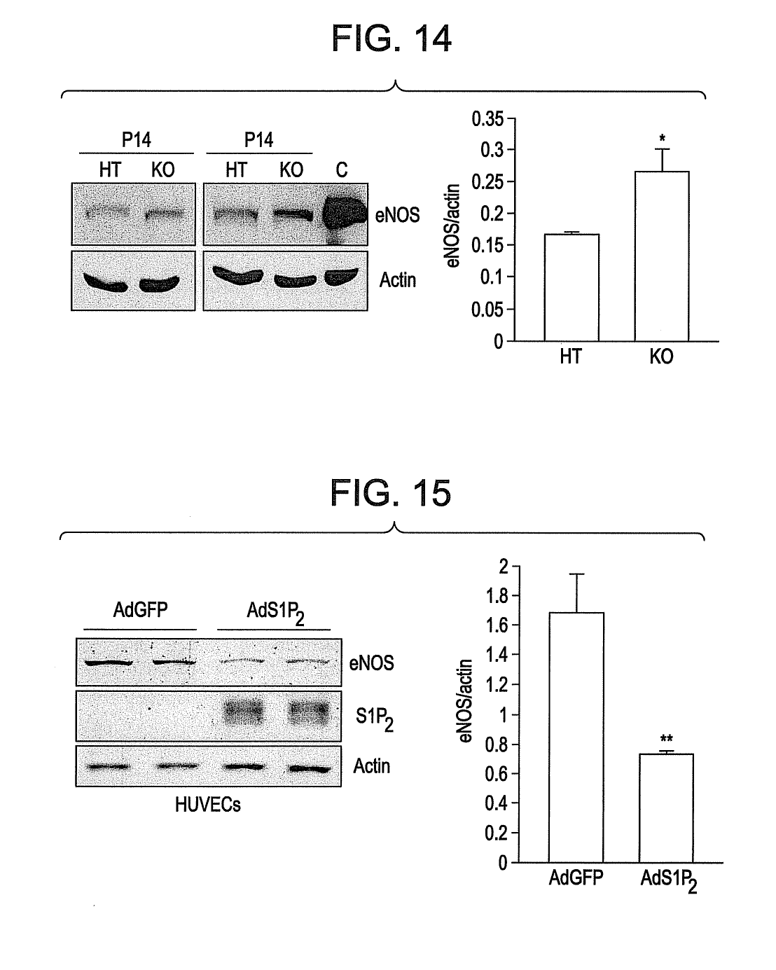

Ischemia-dependent angiogenesis induces eNOS activation and leads to increased NO release that consequently reduces vascular resistance, improves blood flow and enhances vascular remodeling. To explore the possibility of whether the S1P₂ receptor is able to regulate eNOS function, eNOS protein expression of S1p2^{-/-} and S1p2^{-/-} retinal extracts was examined, at the early onset of hypoxia (P14). Indeed, in S1p2^{-/-} retinal extracts, eNOS protein levels were significantly increased by 1.6-fold compared to S1p2^{+/-} counterparts (FIG. 14). To test whether S1P₂ receptor directly regulates eNOS expression, eNOS levels when the S1P₂ receptor is expressed in vitro were quantified. When S1p2 receptor containing adenovirus was transduced into HUVECs, eNOS protein expression was significantly reduced about 2.3-fold relative to control cells (FIG. 15). The effect is partially blocked by inhibition of the Rho-associated protein kinase (ROCK), a key mediator of S1P₂ signaling (data not shown). These results suggest that the S1P₂ receptor negatively regulates eNOS expression.

### Example 9

### Effects of Endogenous S1P2R Blockade on Adherens Junction Assembly and Vascular Permeability

Because umbilical vein endothelial cells (HUVEC) express high levels of S1P1R and lower levels of S1P2R, the role of endogenous S1P2R on the regulation of adherens junction assembly was examined by using the S1P2R-selective antagonist JTE013. JTE013 specifically blocked S1P2R and not S1P1R signaling in a heterologous expression system (data not shown). In HUVECs, blockade of S1P2R with JTE013 resulted in higher Akt phosphorylation levels after S1P stimulation (data not shown), in agreement with the activation of PTEN by S1P via S1P2R. JTE013 enhanced S1P-induced VE-cadherin translocation to adherens junction sites compared with cells preincubated with vehicle and treated with S1P. (data not shown) In addition, S1P2R blockade inhibited S1P-induced stress fibers and potentiated the ability of S1P to induce cortical actin assembly. These data suggest that activation of endogenous S1P2R in endothelial cells counteracts S1P1R and S1P3R-mediated effects on adherens junction assembly and actin cytoskeleton dynamics.

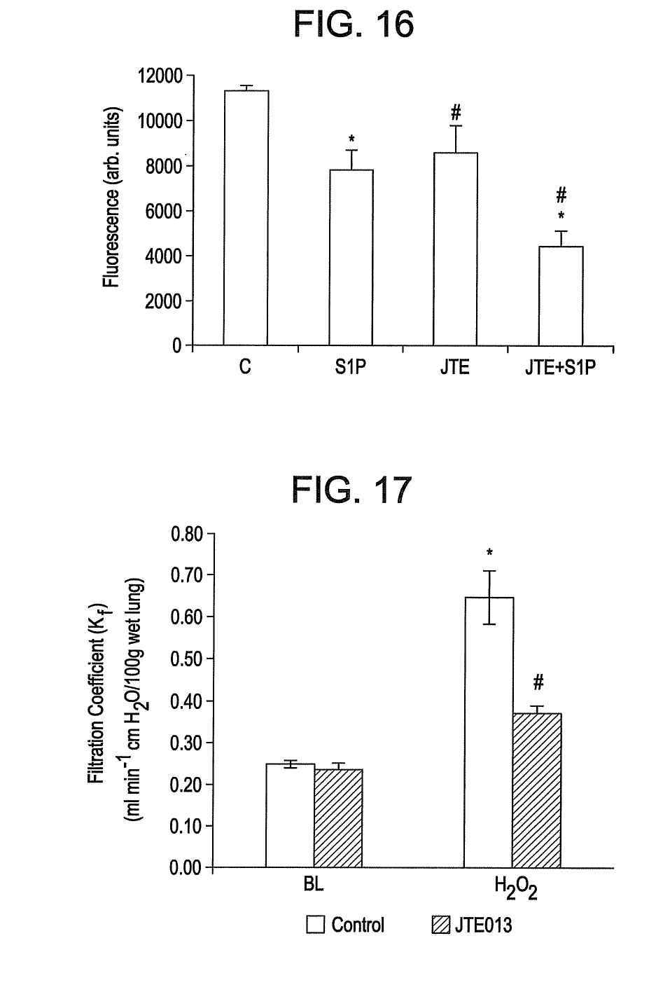

The ability of S1P to enhance barrier properties of HUVEC monolayers after S1P2R blockade with JTE013 was then studied. HUVEC monolayers were incubated with vehicle or with the S1P2R antagonist for 30 minutes. Paracellular permeability in the presence or absence of S1P was measured after 60 minutes. The S1P2R antagonist JTE013 significantly inhibited basal permeability and potentiated the effects of S1P on barrier integrity (FIG. 16). Altogether these data indicate that endogenous S1P2R in HUVEC induced stress fiber formation, disassembly of adherens junctions and increased paracellular permeability.

The effects of the blockade of S1P2R on vascular permeability was further studied in the ex vivo model of perfused rat lungs. Lungs were perfused with either vehicle or 0.5 μmol/L JTE013 during 15 minutes. No significant changes in the capillary filtration coefficient (K_{f}) were induced by these treatments relative to untreated lungs (data not shown). Then, lung edema was induced by addition of 50 μmol/L H₂O₂ to the perfusate. An increase up to 0.647±0.064 in the rate of lung wet weight gain was observed after 15 minutes of H₂O₂ treatment in the vehicle-treated lungs (FIG. 17). Interestingly, in lungs treated with the S1P2R antagonist, H₂O₂-induced lung edema was markedly inhibited (K_{f}=0.374±0.013). These data indicate that the inhibition of S1P2R signaling results in decreased vascular permeability and H₂O₂-induced lung edema.

### Example 10

### Inhibition of S1P2 Receptor and Retinal Vascularization

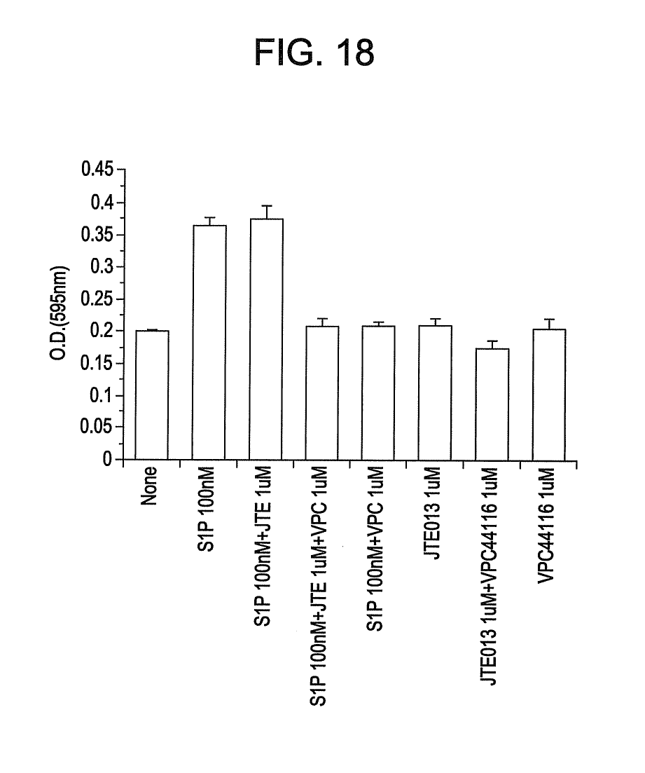

Bovine retinal endothelial cells were tested to see if JTE013, a specific S1P2 receptor antagonist, would influence normal endothelial cell functions, such as cell migration stimulated by S1P. As shown in FIG. 18, JTE013 did not inhibit the ability of S1P to potently induce endothelial cell migration. In contrast, VPC44116, a specific inhibitor of S1P1 receptor blocked normal endothelial cell migration stimulated by S1P in these cells. These data suggest that S1P2R inhibition does not adversely affect normal endothelial cell function, but rather blocks inflammatory events and abnormal vascular tuft formation.

The inventors herein have discovered that the S1P2 receptor is a novel target for the prevention and/or treatment of vision-threatening retinopathies. Antagonists of the S1P2 receptor are suitable for novel compositions and methods for inhibiting abnormal angiogenesis in the eye, particularly in the retina. The compositions and methods are particularly beneficial for the treatment of ocular neovascular disease and neoplastic eye disease.

The terms “a” and “an” do not denote a limitation of quantity, but rather denote the presence of at least one of the referenced item.

The term “or” means “and/or”.

The terms “comprising”, “having”, “including”, and “containing” are to be construed as open-ended terms (i.e., meaning “including, but not limited to”).

The endpoints of all ranges directed to the same component or property are inclusive and independently combinable.

Unless defined otherwise, technical and scientific terms used herein have the same meaning as is commonly understood by one of skill in the art to which this invention belongs.

“Alkyl” is a branched or straight chain saturated aliphatic hydrocarbon group, having the specified number of carbon atoms, generally from 1 to about 12 carbon atoms. The term C₁-C₄alkyl as used herein indicates an alkyl group having from 1 to about 4 carbon atoms. Other embodiments include alkyl groups having from 1 to 8 carbon atoms, 1 to 6 carbon atoms or from 1 to 2 carbon atoms, e.g., C₁-C₈ alkyl, C₁-C₆ alkyl, and C₁-C₂ alkyl.

“Alkoxy” indicates an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups include, for example, methoxy groups.

“Cycloalkyl” indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane. A bicyclic cycloalkyl” is a saturated bicyclic group having only carbon ring atoms. Bicycloalkyl groups have 7 to 12 carbon ring atoms. Examples of bicycloalkyl groups include s-endonorbornyl and carbamethylcyclopentane.

“Mono- and/or di-alkylamino” indicates secondary or tertiary alkyl amino groups, wherein the alkyl groups are as defined above and have the indicated number of carbon atoms. The point of attachment of the alkylamino group is on the nitrogen. The alkyl groups are independently chosen. Examples of mono- and di-alkylamino groups include ethylamino, dimethylamino, and methyl-propyl-amino.

The term “heterocycle” indicates a 5-6 membered saturated, partially unsaturated, or aromatic (“aromatic heterocycle”) ring containing from 1 to about 4 heteroatoms chosen from N, O, and S, with remaining ring atoms being carbon or a 7-10 membered bicyclic saturated, partially unsaturated, or aromatic heterocylic ring system containing at least 1 heteroatom in the two ring system chosen from N, O, and S and containing up to about 4 heteroatoms independently chosen from N, O, and S in each ring of the two ring system. Unless otherwise indicated, the heterocyclic ring may be attached to its pendant group at any heteroatom or carbon atom that results in a stable structure. When indicated the heterocyclic rings described herein may be substituted on carbon or on a nitrogen atom if the resulting compound is stable. A nitrogen atom in the heterocycle may optionally be quaternized. It is preferred that the total number of heteroatoms in a heterocyclic groups is not more than 4 and that the total number of S and O atoms in a heterocyclic group is not more than 2, more preferably not more than 1. Examples of heterocyclic groups include, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benzo[b]thiophenyl, isoquinolinyl, quinazolinyl, quinoxalinyl, thienyl, isoindolyl, dihydroisoindolyl, 5,6,7,8-tetrahydroisoquinoline, pyridinyl, pyrimidinyl, furanyl, thienyl, pyrrolyl, pyrazolyl, pyrrolidinyl, morpholinyl, piperazinyl, piperidinyl, pyrrolidinyl, phthalazinyl, oxazolyl, indolizinyl, indazolyl, benzothiazolyl, benzimidazolyl, benzofuranyl, benzoisoxolyl, dihydro-benzodioxinyl, oxadiazolyl, thiadiazolyl, triazolyl, tetrazolyl, oxazolopyridinyl, imidazopyridinyl, isothiazolyl, and naphthyridinyl.

“Halo” or “halogen” indicates fluoro, chloro, bromo, and iodo.

“Perhaloalkyl” as used herein refers to alkyl groups perhalogenated with fluoro, chloro, bromo, iodo, or a combination of the foregoing halogens.

A dash (“-”) that is not between two letters or symbols is used to indicate a point of attachment for a substituent. For example, —(CH₂)C₃-C₇cycloalkyl is attached through carbon of the methylene (CH₂) group. A dash with a broken line above it indicates the bond can either be a single or double bond.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All cited patents, patent applications, and other references are incorporated herein by reference in their entirety.

1. A method of inhibiting pathological angiogenesis in the eye of a subject in need thereof, comprising
administering to the subject a pharmaceutically effective amount of an inhibitor of the receptor activity of the S1P2 receptor.

2. The method of claim 1, wherein the inhibitor is an antisense RNA, an siRNA, an antibody, or a small molecule.

3. The method of claim 1, wherein the inhibitor is a small molecule of formula I: 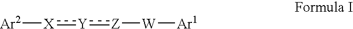
wherein
Ar¹ is an optionally substituted heterocycle or aromatic heterocycle;
Ar² is an optionally substituted heterocycle or aromatic heterocycle;
W is —NR^{a}—, O or —CH₂— wherein R^{a} is hydrogen or C₁-C₃ alkyl;
Z is —C(═O)—, —C(═S)—, O, —CH₂—, ═N—, or ═CH—;
Y is —NR^{a}—, —C(═O)—, —N═, —CH═, ═N—, or ═CH—; and
X is —NR^{a}—, —N═, —CH═, or —CH₂—.

4. The method of claim 3, wherein the antagonist is a small molecule of Formula II: 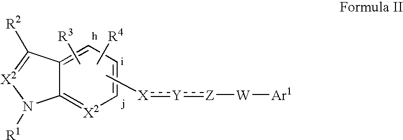
Ar¹ is an aromatic heterocycle;
R¹ is C₁-C₁₂ alkyl;
R², R³, and R⁴ are each independently hydrogen, halogen, C₁-C₆ alkyl, C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;
R³ and R⁴ are optionally positioned at h, i, or j, but not simultaneously at the same position; and
X² is N or —CR^{b}—, wherein R^{b} is hydrogen, halogen, C₁-C₆ alkyl, C₁-C₄alkoxy, C₁-C₆ perhaloalkyl, C₁-C₄ perhaloalkoxy, amino, mono- or di- C₁-C₄alkylamino, C₃-C₇cycloalkyl, or C₃-C₇cycloalkyloxy.

5. The method of claim 4, wherein the antagonist is a small molecule of Formula III: 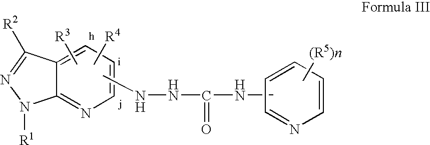
each instance of R⁵ is halogen, C₁-C₆ alkyl, C₁-C₄alkoxy, C₁-C₆ perhaloalkyl, C₁-C₄ perhaloalkoxy, amino, mono- or di- C₁-C₄alkylamino, C₃-C₇cycloalkyl, or C₃-C₇cycloalkyloxy; and
n is 0, 1, 2, 3, or 4.

6. The method of claim 5, wherein R¹ is C₁-C₃ alkyl; R² is C₁-C₃ alkyl, R³ is at position h and is C₁-C₆ alkyl; R⁴ is hydrogen; R⁵ is halogen; and n is 2.

7. The method of claim 1, wherein the pathological angiogenesis is associated with an ocular neovascular disease.

8. The method of claim 7, wherein the ocular neovascular disease is related to age-related macular degeneration, diabetic retinopathy, retinopathy of prematurity, corneal graft rejection, neovascular glaucoma, or retrolental fibroplasia in the subject.

9. The method of claim 8, wherein the ocular neovascular disease is related to age-related macular degeneration.

10. The method of claim 8, wherein the ocular neovascular disease is related to diabetic retinopathy.

11. The method of claim 1, wherein the pathological angiogenesis is the result of an eye injury.

12. The method of claim 1, wherein the pathological angiogenesis is associated with a neoplastic eye disease.

13. The method of claim 1, wherein the pathological angiogenesis of the eye is pathological angiogenesis of the retina or the cornea.

14. The method of claim 1, wherein the inhibitor is in the form of an ophthalmically acceptable formulation comprising an ophthalmically acceptable excipient.

15. A composition suitable for ophthalmic administration, comprising an inhibitor of the receptor activity of the S1P2 receptor and an opthalmically acceptable excipient,
wherein the inhibitor is a small molecule of formula I: 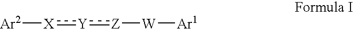
wherein
Ar¹ is an optionally substituted heterocycle or aromatic heterocycle;
Ar² is an optionally substituted heterocycle or aromatic heterocycle;
W is —NR^{a}—, O, or —CH₂— wherein R^{a} is hydrogen or C₁-C₃ alkyl;
Z is —C(═O)—, —C(═S)—, O, —CH₂—, ═N—, or ═CH—;
Y is —NR^{a}—, —C(═O)—, —N═, —CH═, ═N—, or ═CH—; and
X is —NR^{a}—, —N═, —CH═, or —CH₂—.

16. The composition of claim 15, wherein the antagonist is a small molecule of Formula II: 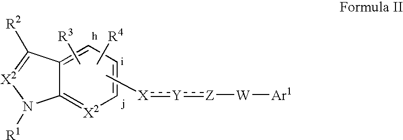
Ar¹ is an aromatic heterocycle;
R¹ is C₁-C₁₂ alkyl;
R², R³, and R⁴ are each independently hydrogen, halogen, C₁-C₆ alkyl,
C₁-C₄alkoxy, C₁-C₆ perhaloalkyl, C₁-C₄ perhaloalkoxy, amino, mono- or di-C₁-C₄alkylamino, C₃-C₇cycloalkyl, or C₃-C₇cycloalkyloxy;
R³ and R⁴ are optionally positioned at h, i, or j, but not simultaneously at the same position; and
X² is N or —CR^{b}—, wherein R¹ is hydrogen, halogen, C₁-C₆ alkyl, C₁-C₄alkoxy, C₁-C₆ perhaloalkyl, C₁-C₄ perhaloalkoxy, amino, mono- or di- C₁-C₄alkylamino, C₃-C₇cycloalkyl, or C₃-C₇cycloalkyloxy.

17. The composition of claim 16, wherein the antagonist is a small molecule of Formula III: 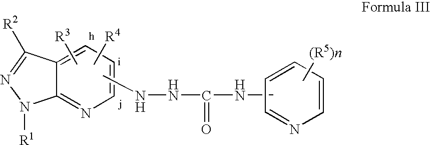
each instance of R⁵ is halogen, C₁-C₆ alkyl, C₁-C₄alkoxy, C₁-C₆ perhaloalkyl, C₁-C₄ perhaloalkoxy, amino, mono- or di- C₁-C₄alkylamino, C₃-C₇cycloalkyl, or C₃-C₇cycloalkyloxy; and
n is 0, 1, 2, 3, or 4.

18. The composition of claim 17, wherein R¹ is C₁-C₃ alkyl; R² is C₁-C₃ alkyl, R³ is at position h and is C₁-C₆ alkyl; R⁴ is hydrogen; R⁵ is halogen; and n is 2.

19. The composition of claim 15, in the form of a topical composition.

20. A method of screening candidate molecules as potential inhibitors of an S1P2 receptor, comprising
contacting S1P2 receptor-expressing retinal endothelial cells in culture with a candidate molecule,
measuring the increase and/or induction of vascular or paracellular permeability in the retinal endothelial cells,
determining if the candidate molecule is an inhibitor of an S1P2 receptor, and producing the molecule.


SEQUENCE LISTING (23 sequences)

SEQ ID NO: 1 - DNA, 20 bp, Artificial Sequence
  PRIMER
atggtgtccactagcatccc

SEQ ID NO: 2 - DNA, 23 bp, Artificial Sequence
  PRIMER
cgatgttcaacttgcctgtgtag

SEQ ID NO: 3 - DNA, 21 bp, Artificial Sequence
  PRIMER
atgggcggcttatactcagag

SEQ ID NO: 4 - DNA, 20 bp, Artificial Sequence
  PRIMER
gcgcagcacaagatgatgat

SEQ ID NO: 5 - DNA, 20 bp, Artificial Sequence
  PRIMER
gcctagcgggagagaaacct

SEQ ID NO: 6 - DNA, 19 bp, Artificial Sequence
  PRIMER
ccgactgcgggaagagtgt

SEQ ID NO: 7 - DNA, 22 bp, Artificial Sequence
  PRIMER
tcaacagcttgctgaccatgat

SEQ ID NO: 8 - DNA, 24 bp, Artificial Sequence
  PRIMER
ggtttgctcttctttacggatagc

SEQ ID NO: 9 - DNA, 23 bp, Artificial Sequence
  PRIMER
cacgacagaaggagagcagaagt

SEQ ID NO: 10 - DNA, 22 bp, Artificial Sequence
  PRIMER
ttcgctggtagacatccatgaa

SEQ ID NO: 11 - DNA, 25 bp, Artificial Sequence
  PRIMER
gaggaggatgagggtgtctataggt

SEQ ID NO: 12 - DNA, 23 bp, Artificial Sequence
  PRIMER
gtgatcagctccaggtttgactt

SEQ ID NO: 13 - DNA, 22 bp, Artificial Sequence
  PRIMER
gggccaccacgctcttctgtct

SEQ ID NO: 14 - DNA, 22 bp, Artificial Sequence
  PRIMER
gccactccagctgctcctccac

SEQ ID NO: 15 - DNA, 21 bp, Artificial Sequence
  PRIMER
tggccaccttgttcagctacg

SEQ ID NO: 16 - DNA, 22 bp, Artificial Sequence
  PRIMER
gccaaggccaaacacagcatac

SEQ ID NO: 17 - DNA, 22 bp, Artificial Sequence
  PRIMER
gtacccggactggattctatgg

SEQ ID NO: 18 - DNA, 21 bp, Artificial Sequence
  PRIMER
gggtgggcttcagcagtaatt

SEQ ID NO: 19 - DNA, 20 bp, Artificial Sequence
  PRIMER
atggcaaatgctggaccaaa

SEQ ID NO: 20 - DNA, 18 bp, Artificial Sequence
  PRIMER
tgccatccagccattcag

SEQ ID NO: 21 - DNA, 26 bp, Artificial Sequence
  PRIMER
caactacatggtctacatgttccagt

SEQ ID NO: 22 - DNA, 17 bp, Artificial Sequence
  PRIMER
tgacccgtttggctcca

SEQ ID NO: 23 - DNA, 3589 bp, Homo sapiens
cggccgccctggggacgcagacgccaaggcccctccggccagggccgggagccgggccggcctagccagttctgaaagccccatggccccagcaggcctctgagccccaccatgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtctgagggtgggggtggaccaacaaccaggccagggcagaggggttcatggagaggccactgggtgaccccagatagagacttggggctactgagccagatgcccccgccccacagacctgggtgatgttgcaaatatttcacacctggaaaggccagataaggcactgactagtcacatagcagtgttgcagtgcggtcctgagggccagtccagtggctagtgtgacccctttagaactggatcctggggaggccagggcaggggacctgtgaagagccagggtgagggcaggcagcatttaaggggagctcagggcaggagcactttaccacctggtacaaaggatttttttttttttttgagacggaatcttgcactgctgcccaggctggagtgcagtggcgtgatctcggctcaccgcaagctccgcctcctgggttcatgtcgttctcctgcctcagcctcccaagtagctgggactataggcgcctgccaccacacctggctaattttttgtacctttagtagagatggggtttcaccgtgttagccaggatggtcttgatctcctgacctcgtgatccgcccgcctcggcctcccaaagtgctgggattacaggcgtgagccaccgtgcccggctttttttttttttttttttttttttttttttttttttgagatgaagtctcgctctgttgcccaggctggagagtgcagtggtacggtctcagctcactgcaacctccacctcccaggttcaagcgattctccagcctgagcctcctgagtagctgggattacaggtgcctaccaccacgcccaggtaattttttttttttttgtatttttagtagagacggggtttcaccatgttggccaggctggtctcgaactcctgacctcatgatccgcccgtgttggcctcccaaagtgtgggattacaggcgtaagccacctcacctggcggtacaaagaatttctgcattttcttccctggcccctagtcctgcaccgatttctccttttcgaatgtattcctcctgccaccttctctgggcaacttcgtgcgactacagaaccactgtcctgaggagctagaggcctcctctctgaccatccagagcccaaatccacagcttccccaaatttcatcagctgccacttgacgacttctccccgtctctctgaggcccggaaaccacggctggaggtggggaggggatggcggctgaggtccattcctcattctcagacctcattgctcagttgcactatttggggcacagaataatcaccaaaagtgagaaaaacgagtttgggtggctggggaggactttgggactcttgatgcaaggcgcaacttgagaaaattctgggtgtgatatttgcacagacaccctcctttcaaaaacagccaccccccaagctattctcagctccacacctgcagccccagctaaggtaccaggtctcctgagcaaggcagagagaagccttgagccttctctgtgtcttctttcaagaaccccgctgtgtcttctttcaagattttttttttgagacagtttcaagatttttgttttgtttttgagatggagtctcactgtgtcacccaggctgaggtggcagtggttcaatctccgttcactgccacctccacctcccgggttcaagcgattctcctgcttcagcctctcgagtagctgggactacaggcacctgccaccatgtctggctaatttttgtatttttagtagagacagggtttcactacgttggccaggctggtctcaaactcctgacctcaagtgatccgcccgcctcggcctccccaattgctgggattacaggcgtgagccactgtgcccggccttcttctttcaagttatatagaatggagcatgggggtggcagtggctagggacatttcctggggacactctcccctaaccccccagaaggacttcacaaaaacctgtggataatggaagggatgttacggtacaaacgtatatttatgtgtgtgtgtgtgtatgtgtgtgcgcgcgcgcgtgtgcacataggcgtgatgtctgtgaccctcctctcctcgtcacatttcccccagaatgaatgctgtcctgtctgctcatgtttgtgttgaagctgccaaagtcggggagctctggtcctgcccagacccctttggaattgctggcccatcctcccactggagagctggggtgcagctcaccttggggaaggaaacctcatgcctcagagtaatttcttgtgaatgcaaagcctgggggagcgggtctttggggggcaaggagccagtcaggggcttgtttcccctcatagagctccccagacgtgcctccgcaatgcctgaaacccagacctaggctaataaacggttcaatttctgttaaaaa